(19) United States
(12) Patent Application Publication
(10) Pub. No.: US 2016/0258954 A1
(43) Pub. Date: Sep. 8, 2016

(54) METHOD OF DIAGNOSING AND TREATING EPSTEIN BARR VIRUS-BASED MYALGIC ENCEPHALOMYELITIS CHRONIC FATIGUE SYNDROME PATIENTS USING A SERUM ANTIBODY ASSAY

(71) Applicant: CFS, LLC, Beverly Hills, MI (US)

(72) Inventor: A. Martin LERNER, Birmingham, MI (US)

(21) Appl. No.: 15/012,352

(22) Filed: Feb. 1, 2016

Related U.S. Application Data
(60) Provisional application No. 62/110,562, filed on Feb. 1, 2015.

(51) Int. Cl. G01N 33/569; A61K 31/4178; A61K 31/675; A61K 31/195

(52) U.S. Cl. CPC G01N 33/56994; A61K 31/195; A61K 31/4178; A61K 31/675; G01N 2333/05

(57) ABSTRACT

A method of diagnosing a subset of Epstein Barr Virus, Myalgic Encephalomyelitis Chronic Fatigue Syndrome (ME/CFS) patients through a multi-prong clinical/serological analysis is provided wherein Epstein Barr Virus Abortive Lytic Replication (EBV) is determined by detecting the presence of BMRF1 in serum antibody assays. A method of treating patients diagnosed with Epstein Barr Virus Abortive Lytic Replication (EBV), Myalgic Encephalomyelitis Chronic Fatigue Syndrome (ME/CFS) with specific antiviral nucleosides is also provided, to alleviate the condition.

### CROSS-REFERENCE TO RELATED APPLICATIONS

This application claims the benefit of U.S. provisional application Ser. No. 62/110,562 filed Feb. 1, 2015 the disclosure of which is hereby incorporated in its entirety by reference herein.

### TECHNICAL FIELD

The present invention relates to a method of classifying, diagnosing and treating a subset of Epstein Barr Virus, Myalgic Encephalomyelitis Chronic Fatigue Syndrome (ME/CFS) or Systemic Exertion Intolerance Disease (SEID), patients. In particular, the invention relates to a protocol for classifying an appropriate subset of patients through a multi-prong clinical/serological analysis, diagnosing patients with Epstein Barr virus (EBV) as the specific causal agent of chronic fatigue syndrome through the use of an antibody to EBV DNA Polymerase processivity factor, BMRF1, as a molecular marker and further treating diagnosed patients with specific antiviral nucleosides which alleviate the condition.

### BACKGROUND

Chronic Fatigue Syndrome (CFS), also known as myalgic encephalomyelitis (ME) (recently proposed to be renamed SEID by the Institute of Meeicine, and post viral fatigue syndrome, is a life altering illness with inflammatory and neurocognitive symptoms affecting women to men in a ratio of 4:1. To date, evidence-based etiology or treatment has been elusive. CFS manifestations are life-altering fatigue in ordinary activities, including constellations of syncope, chest pain, muscle aches, palpitations, sore throat, low-grade fevers, and inability to exercise without a worsening of symptoms, cervical lymphadenopathy, cognitive impairment and resultant depression.

ME/CFS is not rare. Current estimates provide that there are as many as four million persons in the United States alone who have CFS-like symptoms. However, the disorder remains debilitating, complex and mysterious in origin, natural history, understanding and treatment.

The spontaneous recovery rate for CFS patients is low, for example, 19%. Numerous treatment regimens have been proposed and include administration of various agents such as immune stimulators and steroids, as well as recommending exercise and psychiatric treatment. While they may lead to modest short-term improvement, such treatments have proven generally ineffective in the long run. As the underlying causes and distinctions among types of CFS patients have not previously been known, both observational and evidence-based trials have been misdirected or inappropriately planned.

While progress has been made to segregate certain groups of CFS patients and provide them with specific antiviral agents to alleviate the condition, for other CFS patients—namely those found to have herpes virus plus co-infections—no effective treatment option has been identified to date.

Accordingly, given the distinct types of CFS patients, underlying causative agents and varying treatment approaches, there exists a need for a methodology to identify the appropriate subset of myalgic encephalomyelitis chronic fatigue syndrome patients, a serological method to diagnose this subset and confirm the causative agent involved with a reliable diagnostic assay. With the help of a diagnostic assay, a specific treatment protocol can be implemented to alleviate the CFS symptoms in these patients and restore their ability to lead a normal or near-normal life, free from the debilitating effects of chronic fatigue.

### SUMMARY

In one embodiment of the invention, a method of diagnosing an Epstein-Barr virus subset of Myalgic Encephalomyelitis-CFS patients is disclosed, including the step of Identifying Epstein-Barr virus Abortive Lytic replication in patients with Myalgic Encephalomyelitis-Chronic Fatigue Syndrome by determining the presence of EBV early encoded DNA Polymerase processivity factor, BMRF1.

In another embodiment, a method of diagnosing an Epstein-Barr virus subset of Myalgic Encephalomyelitis-Chronic Fatigue Syndrome patients is disclosed and includes the steps of: 1) Determining the presence of encoded EBV Early Antigen, Diffuse; 2) Determining the presence of EBV encoded DNA polymerase; and 3) Diagnosing a patient with Epstein-Barr Abortive Lytic Replication when EBV Early Antigen, Diffuse is found in conjunction with the presence of EBV encoded DNA polymerase.

In an additional embodiment, a method of diagnosing the causation agent for a Myalgic Encephalomyelitis-Chronic Fatigue Syndrome patient is disclosed and includes the step of: Determining the absence of Epstein-Barr virus Abortive Lytic replication, through the following sub-steps: 1) Determining the absence of EBV VCA IgM; and 2) Determining the absence of EBV encoded DNA polymerase through assays for serum antibodies to the early EBV non-structural proteins of the EBV tegument.

In yet another embodiment, a method of diagnosing and treating an Epstein-Barr virus subset of patients with Myalgic Encephalomyelitis-Chronic Fatigue Syndrome is disclosed and includes the steps of: 1) Identifying Epstein-Barr virus Abortive Lytic replication by using the serum antibody to EBV-encoded DNA Polymerase processivity factor, BMRF1, as a molecular marker; and 2) Treating the subset of patients with the administration of a therapeutically effective amount of at least one antiviral agent.

### BRIEF DESCRIPTION OF THE DRAWINGS

FIG. 1 is a table outlining the CFS Energy Index Point Score Metric;

FIG. 2A is a table depicting the clinical results that establish EBV Abortive Lytic replication in the first of six EBV subset ME/CFS patients treated with valacyclovir for over 12 months;

FIG. 2B is a table depicting the clinical results that establish EBV Abortive Lytic replication in the second of six EBV subset ME/CFS patients treated with valacyclovir for over 12 months;

FIG. 2C is a table depicting the clinical results that establish EBV Abortive Lytic replication in the third of six EBV subset ME/CFS patients treated with valacyclovir for over 12 months;

FIG. 2D is a table depicting the clinical results that establish EBV Abortive Lytic replication in the fourth of six EBV subset ME/CFS patients treated with valacyclovir for over 12 months;

FIG. 2E is a table depicting the clinical results that establish EBV Abortive Lytic replication in the fifth of six EBV subset ME/CFS patients treated with valacyclovir for over 12 months;

FIG. 2F is a table depicting the clinical results that establish EBV Abortive Lytic replication in the sixth of six EBV subset ME/CFS patients treated with valacyclovir for over 12 months;

FIG. 3 is a table depicting the clinical results that provide EBV Abortive Lytic Replication in twenty Comparison Group patients; and

FIG. 4 is a flow chart depicting the steps associated with diagnosing a patient with ME/CFS.

FIG. 5 illustrates a Reference Serum Test showing the signal differential between the negative and positive serum samples.

FIG. 6 illustrates an EBV Antigen Negative Control Test, which demonstrates that all assays are negative when a confirmed EBV negative control is used.

FIG. 7 illustrates a Healthy Serum Test, which demonstrates that unlike the other antigens tested BMRF1 is specific when compared with serum from healthy individuals.

FIG. 8 illustrates a reference range for the standard curve.

### DETAILED DESCRIPTION

Reference will now be made in detail to embodiments and methods of the present invention which constitute the best modes of practicing the invention presently known to the inventors. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary, percent (%), “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a”, “an”, and “the” comprise plural referents unless the context clearly indicates otherwise. For example, reference to a component in the singular is intended to comprise a plurality of components.

Throughout this application, where publications are referenced, the disclosures of these publications in their entireties are hereby incorporated by reference into this application in their entirety to more fully describe the state of the art to which this invention pertains.

The term Chronic Fatigue Syndrome (CFS), Myalgic Encephalomyelitis Chronic Fatigue Syndrome (ME/CFS) and Systemic Exertion Intolerance Disease (SEID) are used synonymously herein. As used herein, CFS is defined to be a disorder caused by infection with a CFS-causing agent. “CFS-causing agent” includes CFS-inducing herpes viruses, for example, EBV, human cytomegalovirus (HCMV), and/or Human herpes virus 6 (HHV6). Based on the CFS-causing agent—2 distinct patient classifications have been created. For group A patients the CFS-causing agent is a CFS-inducing herpes virus such as EBV, HCMV and HHV6. For group B patients, with specific co-infections (such as _tick-borne infections) the encoded proteins may circulate by the blood stream and peripheral area, causing end-organ injury (e.g. heart, muscle, brain, liver, etc.)

The term “diagnosing: encompasses, for example, characterizing a CFS patient as belonging to a particular predefined subset of CFS causal groupings;

The term “treatment” refers to the prevention, partial alleviation or cure of the condition or disorder, or at least one symptom of the condition or disorder.

The term “effective’ or “therapeutically effective” means sufficient to cause at least one of a patient's symptoms to decrease in frequency and/or intensity. To this end, one measure for effectiveness is the Energy Index point score (EIPS), which monitors the course of recovery of CFS patients under treatment by observing an increase in the EIPS of 1.0 or more units, and/or a decrease in serological indices of pathogens.

The term “infection” means the invasion of a host organism's body by another organism or entity, for example, a virus or bacteria. Infection by a virus may, but does not necessarily, include entry of the virus into host cells, production of gene products based on the viral nucleic acid, replication of the virus, and/or further spread of the virus within the host body, which may or may not induce an immunological response by the host organism. “Infection” may include the latent presence of virus, for example, that which is not replicating, and whose genes are not being expressed; or, more typically, “infection” may include a virus, at least some of whose genes are being transcribed into mRNA, which may be translated into protein gene products.

“Infection” includes abortive infection and/or replication. As used herein, “abortive” refers to infections characterized by incomplete viral replications, for example, with non-assembly into a complete virion. Abortive infection can include, for example, expression of the virus genome to produce early (IE), middle (E) or late (L) gene products including, for example, EBV encoded DNA polymerase. In such an example of abortive infection, the gene products are not assembled into a complete virus. Abortive infection may include, primarily or exclusively, early only, early and middle, or early, middle and late gene products. Abortive herpes virus replication is a proposed pathogenic mechanism of CFS that can be used to diagnose the disease, and to identify patients who are good candidates for antiviral therapy.

The term “primary abortive replication” includes, for example, first episode infection with EBV, HCMV and HHV6. The term “primary abortive replication” and “primary infection” are substantially equivalent terms.

The term “co-infection” includes infection with, for example, Borrelia burgdorferi, Streptococcus pyogenes, Ehrlichia chaffeensis, Babesia microti and Mycoplasma pneumoniae.

“Secondary nonviral infectious agent” includes, for example, Borrelia burgdorferi, Streptococcus pyogenes, Ehrlichia chaffeensis, Babesia microti and Mycoplasma pneumoniae. Streptococcus pyogenes infection may manifest itself as Adult Rheumatic Fever. As used herein “secondary infectious agent” and “secondary nonviral infectious agent” are substantially equivalent terms.

The term “BMRF1” or “EBV DNA polymerase processivity factor” refers to a non-structural viral gene product from EBV, which is also sometimes also called diffuse early antigen [EA-D].

As used herein, “antiviral agent” includes, for example, valacyclovir, valganciclovir, maribavir, cidofovir, famciclovir and foscarnet. However, any antiviral agent that is effective against a CFS-inducing infection can be used according to the methods disclosed herein.

The amount of antiviral agent required to constitute a therapeutically effective amount will vary based on a number of factors, including the severity of the chronic fatigue syndrome; the identity, age, body weight, general health, gender, diet and chemical make-up of the patient; the type and degree of the cellular response to be achieved; the specific agents or composition employed, and its activity; the time of administration, route of administration, range of excretion of the agent; the duration of the treatment; drugs used in combination or coincidental with the specific agent; and like factors well-known in the medical arts. It must also take into consideration the therapeutic window, that is, the need to adjust and minimize toxic side-effects. For example, it is well within the skill of the art to start doses of the agents at levels lower than those required to achieve the desired therapeutic effect and to gradually increase the dosages until the desired effect is achieved.

### Myalgia Encephalomyelitis Chronic Fatigue Syndrome (ME/CFS)

CFS was recognized as a public health problem because of recurrent signs and symptoms of this previously unknown life-altering illness. Cardiac, immune dysfunction, cerebral abnormalities have been identified. The average death age of 144 CFS patients registered by the National CFIDS Foundation was 39.3 years, and 20.1% died of suicide, and 20.1% of heart failure.

CFS is generally defined as a disorder of uncertain cause that is characterized by persistent, profound fatigue, usually accompanied by impairment and short-term memory or concentration, sore throat, tender lymph nodes, muscle or joint pain, and headache unrelated to any preexisting medical condition, that typically has an onset at between the ages of 30-50 of age. Medline Plus Medical Dictionary. CFS is often characterized by abortive replication and an inability on the part of the patient to inactivate the CFS-causing agent by inducing, for instance, inactive herpes virus latency. CFS patients can respond to antiviral therapy, as measured by, for example, a reduction in nucleic acid gene products of one or more CFS-causing agents.

Clinical tests have shown cardiac, immunologic, radiographic and genetic abnormalities in CFS patients. It is believed that CFS is caused by Epstein-Barr virus (EBV), cytomegalovirus (HCMV), and human herpes virus 6 (HHV6) in single or multiple virus infection. This paradigm affirms that the herpes viruses, despite maximum efforts from these immuno-competent affected patients, continue an incomplete abortive replication of middle-gene products, usually without achieving complete virus synthesis. Per this hypothesis, CFS patients are believed to continue EBV, HCMV and HHV6 herpes virus replication, and do not achieve the viral latency necessary for recovery. As such, it was proposed that early and middle herpes virus (EBV, HCMV and HHV6) gene products to about the fiftieth gene of these complex viruses, containing over 200 open-reading frames, are synthesized without achieving complete virus formation. This hypothesis was tested with the nucleosides valacyclovir for a suspected EBV CFS subset and valganciclovir for suspected HCMV or HHV6 CFS subsets. As set forth in the International Application under Publication No. WO 2009/054957, by Dr. A. Martin Lerner, incorporated by reference herein in its entirety, specific methodologies have been disclosed to successfully classify and treat patients with EBV, HCMV and HHV6 in single or multiple infection without co-infection, through the administration of specific antiviral agents or specified treatment periods, as assessed by the validated severity of an illness metric, the Energy Index Point Score (EIPS). Further information pertaining to Dr. Lerner's work in diagnosing and treating CFS through the use of antiviral agents can be found in U.S. Pat. Nos. 5,872,123, 6,258,818, 6,399,622, 6,537,997 and 6,894,056, which are herein incorporated by reference in their entirety.

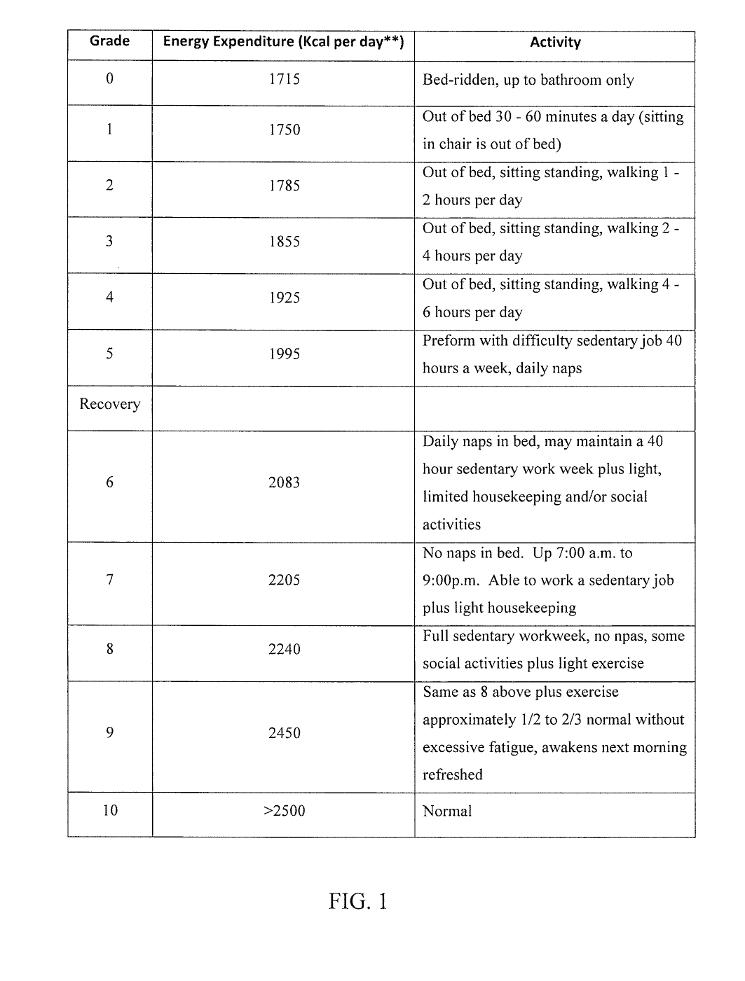

As depicted in FIG. 1, the Energy Index (EI) point score (Copyright, Lerner AM and Deeter R G, 1999), is a simple and reliable metric that can be used to easily evaluate the functional capacity of the CFS patient at each patient-physician visit. *The CFS Energy Index point score can also be determined by questionnaire (US copyright, 1999 Lerner A. M., Deeter, R. J) reproduced with permission. **Kcal per day is calculated for a 70 kg CFS patient. The EI is measured on a scale of 1-10. Validation of the EI was done using two methods: 1.) 20 CFS patients and 22 healthy adults, matched for sex, age, place, and time; EI, CFS=3.6; EI, healthy adults=9.9, p=<0.0001; and 2.) 55 CFS patients evaluated at the same time by the EI and fatigue severity score, correlation 0.67, p=0.0066. Improvement to disappearance of CFS symptoms correlates with an increasing EI.

The validated energy index point score generally can be calculated for each CFS patient every 3 months at physician visits. A CFS patient has an EI≦5. A CFS patient with an EI of 0 is bedridden; a CFS diagnosis is no longer present at an EI>5. The EI effect size is 0.25, a medium effect size is 0.5. A large effect size is >0.8.

While CFS patients classified with EBV, HCMV and HHV6 in single or multiple infections without co-infection have been diagnosed and treated successfully, that is not the case to date for the group of EBV subset CFS patients with co-infections, such as tick-borne Borrelia burgdorferi, Babesia microti, Anaplasma phagocytophila and/or adult rheumatic fever. For this group of patients, their ability to lead fulfilling and productive lives has to-date remained significantly compromised. This invention addresses the long standing, but unmet need to properly diagnose the causative agent of CFS and its groupings, so that an appropriate treatment plan can be administered.

### Epstein-Barr Virus

Epstein-Barr virus (EBV), a gamma herpes virus, is one of the causative agents of the Chronic Fatigue Syndrome. EBV, like other herpes viruses, encodes for several enzymes that are involved in viral DNA replication; all are part of the early antigen (EA) complex. Several EBV-associated enzymes have been described to date, such as thymidine kinase (TK), Deoxyribonucleotide polymerase (DNA polymerase), deoxyribonuclease (DNASE), deoxyuridine Triphosphate Nucleotidohydrolase (dUTPase), and ribonucleotide reductase, as well as uracil-DNA glycosylase. Historically, while these antibodies to EBV-encoded enzymes were observed in patients with different EBV-associated diseases, the reason for these antibody patterns and the role these proteins might play in the pathophysiology of disease, separate from their role in virus replication has been unknown. Some hypotheses were described in an article entitled “Stress-associated changes in the steady-state expression of latent Epstein-Barr virus: Implications for chronic fatigue syndrome and cancer” in Brain, Behavior and Immunity 10 (2005) 90-103, incorporated by reference herein.

### Creation of a ME/CFS Diagnostic Panel & the Identification of Molecular Markers

A ME/CFS diagnostic panel was created utilizing initial Fukuda/Carruthers criteria and a systematic review of 142 ME/CFS patients. Two groups of ME/CFS patients were found. Group A patients had elevated serum IgG antibody titers to the herpes viruses Epstein-Barr virus (EBV), cytomegalovirus (HCMV) and Human Herpes virus 6 (HHV6) in single or multiple virus infection, but no other co-infections. Group B patients had similar elevated herpes virus antibody titers, plus serologic evidence of co-infections, tick-borne (“Borrelia burgdorferi”, “Anaplasma phagocytophilia”, “Babesia microti”); “Mycoplasma pneumoniae”; or adult rheumatoid fever. One hundred and six group A ME/CFS patients were followed from 2001-2007, and treated with subset directed valacyclovir EBV subset or valganciclovir (HCMV, HHV6 subsets). The data included over 7000 patient visits and 35,000 data entries. Seventy-nine (74.5%) of the group A patients recovered to resume normal life (p<0.0001)—the results are unprecedented.

A challenge to understanding Systemic Exertion Intolerance Disease (SEID) is the absence of a diagnostic biomarker such as the isolation of Streptococcus pneumoniae from the blood of a patient with pneumonia. The pneumococcus isolate guides effective treatment. 142 Myalgic Encephalomyelitis/Chronic Fatigue Syndrome (ME/CFS) patients were reviewed (now proposed to be renamed SEID by the Institute of Medicine) in an inclusive investigator-blinded systematic longitudinal study including over 5,000 patient visits and 35,000 data entries. At that time, two groups of “SEID” patients were found. One hundred and six Group A patients had elevated structural serum IgG antibody to viral capsid antigen Epstein-Barr virus (EBV) alone, or with similar elevated antibody titers to cytomegalovirus (CMV) and/or Human Herpesvirus 6 (HHV6). No co-infections were identified in Group A patients. There were no IgM antibodies to EBV, CMV, or HHV6 structural antigens. These data indicate past infection with no current herpesvirus lytic replication. Group B SEID patients had elevated herpesvirus IgG serum titers to structural antigens (EBV, CMV, HHV6) plus elevated serum antibody at least two-times the value of a negative test to co-infections. Co-infections were tick-borne (Borrelia burgdorferi, Anaplasma phagocytophilia, Babesia microti), Mycoplasma pneumoniae or adult rheumatic fever. Notwithstanding the advances made, until now an evidence-based simple test for the diagnosis of ME/CFS has remained elusive. Dr. Glaser in conjunction with Dr. Williams and Dr. Lerner independently suggested that ME/CFS is abortive lytic herpes virus replication without DNAemia, antigenemia or IgM antibody to herpes virus structural antigens. Specifically, it is believed that ME/CFS may be associated with EBV, CMV, HHV6 “abortive” lytic replication resulting in herpesvirus early-encoded non-structural viral proteins in the blood of ME/CFS patients. These early encoded proteins are critical to intracellular viral lytic replication, but they are “ordinarily” limited to intracellular virus-infected cells. During EBV replication, B-lymphcytes and epithelial cells in the tonsils are affected.

Drs. Glaser and Williams found that the early EBV protein dUTPase produced both illness behavior in a murine model, and immunologic abnormalities in peripheral blood mononuclear cells in vitro. Similar immunologic disarray with quantitative changes of intracellular perforins and granzymes were found in ME/CFS patients. Since valacyclovir and valganciclovir do not inhibit early herpes virus proteins, it was suggested that new herpes virus host cell recruitment had been interrupted in the ME/CFS patients who had recovered their health. Valacyclovir after gastrointestinal absorption has a 200× greater affinity for EBV thymidine kinase than for the host cellular enzyme. (1) Valganciclovir inhibits HCMV and HV6 DNA polymerases. As such, it was believed that herpes virus early proteins may be critical in the etiology of ME/CFS.

In this regard in patients with HCMV subset ME/CFS, a unique presence of IgM HCMV early proteins p52 (UL44) and CM2 (UL 44-UL 57) was detected in 61 HCMV subset patients, but these serum antibodies were not found in a comparison group of well patients. It was thus reported that elevated serum antibody titers to EBV (EAD) in 86 of the 106 (81%) ME/CFS patients with group A ME/CFS were found.

It was reported that elevated serum antibodies to a complex of EBV early-encoded proteins in SEID patients which are transiently present in cases of Infectious Mononucleosis and had been named EBV Early Antigen (Diffuse), EA(D). Group A EBV subset SEID patients had elevated serum antibody to EBV EA(D). Additionally, it was reported elevated serum-neutralizing antibodies against the early EBV encoded-protein DNA polymerase (BALF5) and EBV deoxyuridine triphosphate nucleotidohydrolase (dUTPase) (BLLF3). Low-titers of EBV EA(D) were occasionally present in normal controls (eg<30 units), but antibody to EBV early-encoded proteins EBV DNA polymerase and EBV dUTPase were not present in controls. It was suggested that there may be a prolonged presence of elevated antibody to EBV dUTPase and EBV DNA polymerase in EBV subset of SEID patients, and if these preliminary findings were corroborated with larger numbers of patients and appropriate controls, this antibody panel [EBV EA(D), EBV DNA poly, EBV dUTPase] may identify EBV subset SEID patients.

EBV DNA polymerase accessory processivity factor (BMRF1) is complexed with EBV DNA polymerase. BMRF1 function is necessary for EBV DNA polymerase chain lengthening, and is required for EBV lytic replication. Serum antibody to BMRF1 is an identifying biomarker for EBV subset SEID patients. It may be that the apparent discrepancy in prior finding, namely the absence of antibody to BALF5 (DNA polymerase) in this report, is that the earlier presence of apparent antibody to DNA polymerase (BALF5) was due to a BALF5-BMRF1 “unidentified” complex. Earlier studies may have measured antibody to BMRF1 and inadvertently called this antibody to DNA polymerase.

EBV early gene proteins dUTPase and DNA polymerase are enzymes involved in EBV lytic DNA replication. A repetitive presence of positive serum antibodies to EBV encoded gene products dUTPase and DNA polymerase has been found in 6 EBV subset ME/CFS patients. Over a period of 13-16 consecutive months from 2003-2007, serum assays from these 6 EBV subset ME/CFS patients tested positive to EBV dUTPase in 25/50 assays (50%) and to EBV DNA polymerase 40/52 assays (76.9%). Comparison group assays for EBV dUTPase and EBV DNA polymerase from 20 control age-sex matched persons were negative. The presence of the EBV proteins dUTPase and DNA polymerase in the blood of ME/CFS patients indicate abortive lytic replication. Accordingly, the presence of serum antibodies to EBV, dUTPase and EBV DNA polymerase were believed to be diagnostic molecular markers for the EBV subset of ME/CFS patients. Further studies have refined this understanding and as presented below it is now believed that EBV DNA polymerase processivity factor (BMRF1), which is intimately complexed with EBV DNA polymerase, aids with EBV DNAchain lengthening and can serve as the identifying biomarker for EBV subset ME/CFS patients.

### Testing Methods

### Identification of Group A/Group B ME/CFS Patients

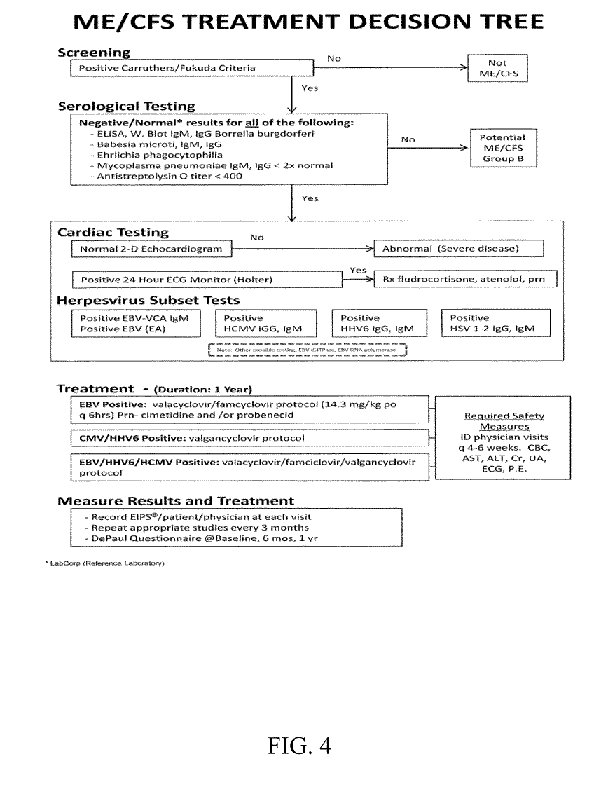

6 ME/CFS patients were identified as Group A EBV subset (five patients), and one patient (group B) had co-infection with Borrelia burgdorferi. The single ME/CFS group B patient had a positive Borrelia burgdorferi western blot IgM test. A ME/CFS treatment decision tree that developed from the systematic review of the 142 ME/CFS patients is provided as FIG. 4.

### Comparison Group

Blood samples were taken from unknown persons at a commercial laboratory. The age and sex of the comparison group were selected to be similar to ME/CFS patients.

EBV, VCA, IgM, VCA p18 peptide is a defined VCA-specific marker protein utilized in the ETI-EBV-M reverse assay (DiaSorin, Inc., Stillwater, Minn., USA). It consists of 56 amino acids of the BFRF encoded VCA and contains immune-dominant epitopes. This ETI-EBV-M reverse kit utilizes the enzyme-linked immunosorbent assay (ELISA) based on the antibody capture technique. The absorbance of the solution measured at 450 nm is related to the concentration of IgM to EBV VCA present in the reaction solution.

EBV-IgG Early Antigen (diffuse), EA(D). The ETI-EA-G kit (DiaSorin) for quantitative detection of IgG antibodies to EBV Early Antigen Diffuse (EBV-EA (D) was used. Diluted serum was incubated with recombinant EA(D) peptide bound to the solid surface of a micro titer well. The ETI-EA-G assay uses an EA(D) 47 KD recombinant polypeptide. The absorbance of the solution, measured at 450 nm is proportional to the concentration of IgG antibodies to EBV EA(D) present in the reaction solution.

HCMV ELISA testing for CMV IgG and CMV IgM was performed using ELISA kits from DiaSorin. The HCMV IgG kit contains purified HCMV strain AD-169 antigen-coated wells. The HCMV IgM ELISA is a microcapture assay with wells coated with anti-human IgM antibody to the same strain AD-169. Sera were diluted 1:10 and incubated for one hour at 37° C. The wells were washed three times in washing buffer and bound HRP label was detected with 3,3′ 5.5 tetramethyl benzidine as substrate for 30 minutes in the dark, after which the color reaction was stopped by the addition of stop solution as recommended by the manufacturer's manual. The absorbance was measured at 450/650 nm using Biotech reader (Biotech Clinical Laboratories, Inc., Farmington Mich., USA).

Neutralization assays (DNA polymerase, DNase, and dUTPase) were performed as previously described. Briefly, 5 μl of human serum were mixed with 5 μl of either purified EBV-encoded dUTPase (3-5 units of enzyme) or an extract from TPA/sodium butyrate induced Raji cells (for EBV-encoded DNA polymerase) for 30 min at room temperature prior to assaying for enzymatic activity. EBV-encoded DNA polymerase and dUTPase activity were determined as described previously. Raji cells were induced by treatment with TPA and sodium butyrate for 48 hrs. Cells (106-8) were harvested, re-suspended in 1 ml of extraction buffer (50 mm Tris-HCl, pH 8.0 2 mM ATP, 0.2 M KCl, 3 mM dithiothreitol, 2 mM MgCl2 0.2 mM phenylmethylsulfonylfluoride and 10% (v/v) glycerol, lysed by sonication and centrifuged at 14,000×g for 5 min. The resulting supernatant was employed for the EBV-encoded DNA polymerase assay. Purified EBV-encoded dUTPase was also obtained as previously described.

For positive controls, assays were performed in the absence of human sera that lacked detectable antibodies to the EBV encoded dUTPase and DNA polymerase and negative controls were performed in the absence of the enzyme preparation. Units neutralized were obtained as follows: (Ucontrol-Userum). Serum with neutralizing units greater than or equal to two standard deviations from the control are considered “positive” for dUTPase or DNA polymerase neutralizing antibodies.

The following tests were performed by Lab Corps (Dublin, Ohio) on the 6 ME/CFS patients (Groups, A 5 patients, Group B, 1 patient) 163600 Lyme, Western Blot and ELISA, serum—IgG and IgM. Method. Antigen—whole-cell proteins were extracted from B. burgdorferi strain B31, resolved by polyacrylamide gel electrophoresis into individual antigen bands and then transferred to nitrocellulose strips for blotting.

138315 Babesia microti Antibody Panel—IgG and IgM. Method—IFA. Antigen—the substrate for the IFA was guinea pig or hamster erythrocytes infected with Babesia microti organisms and then fixed onto microscope slides. Upon interaction with human sera containing anti-Babesia antibodies and the appropriate conjugate, infected cells fluoresce.

164722 Ehrlichia Ab panel “(Granulocytic and Monocytic/Anaplasma phagocytophilia)”—IgG and IgM. Method: IFA. Antigen: is either inactivated HGE or HME

163758 Mycoplasma pneumoniae Antibodies—IgG and IgM. Method: EIA. Antigen: Mycoplasma pneumoniae FH antigen

006031 Antistreptolysin 0 Ab. Method: Latex immunoturbidimetry. Human Antistreptolysin 0 antibodies agglutinate with latex particles coated with streptolysin 0 antigens. The precipate is determined turbidimetrically at 552 nm.

### Results

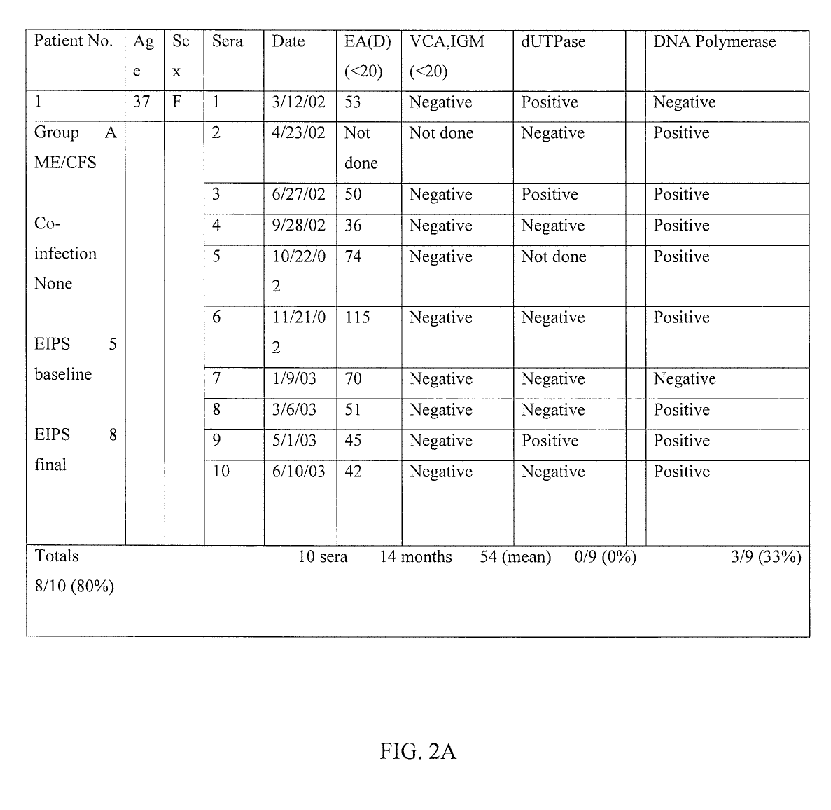

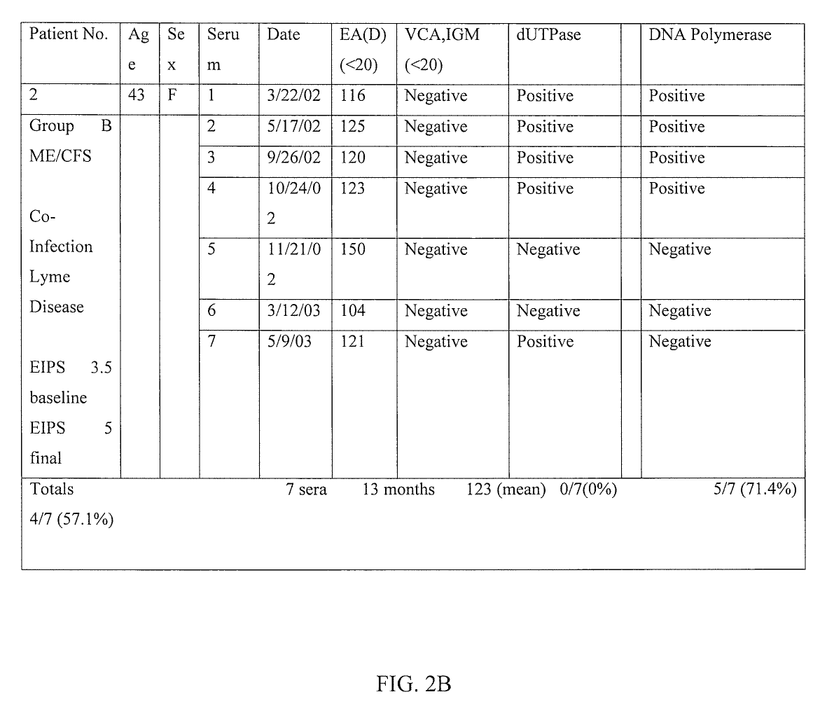

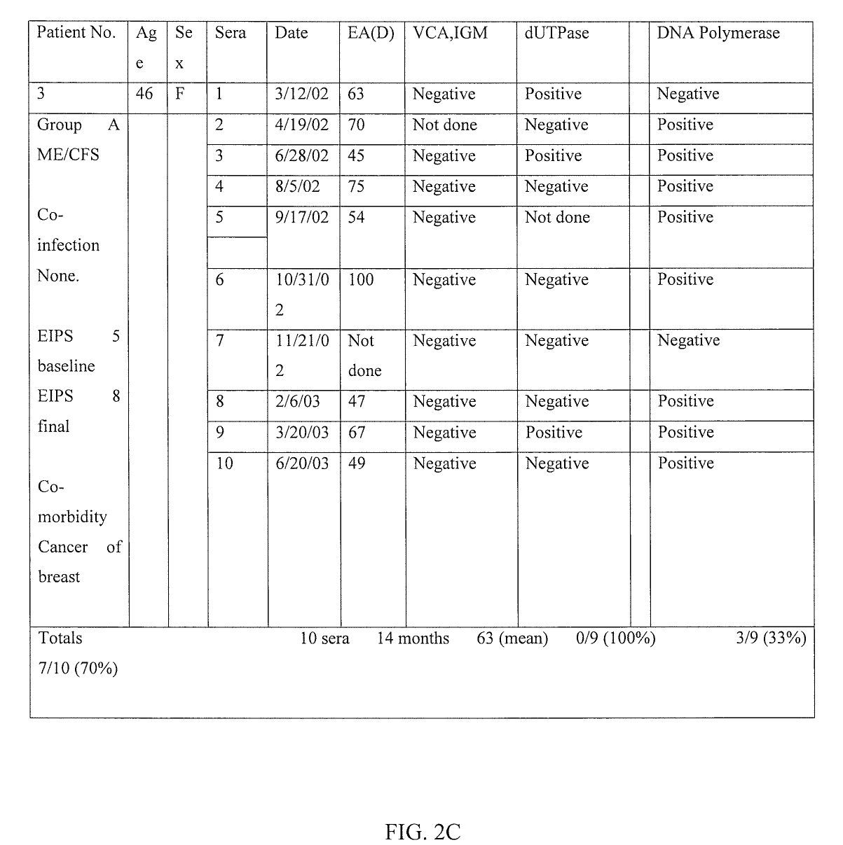

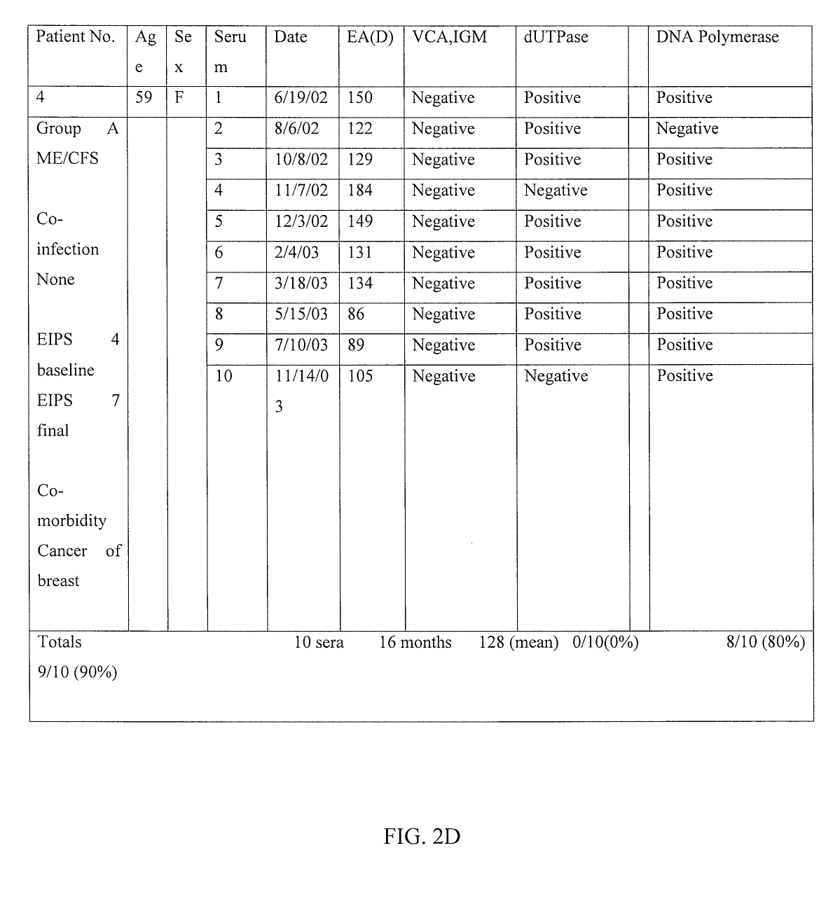

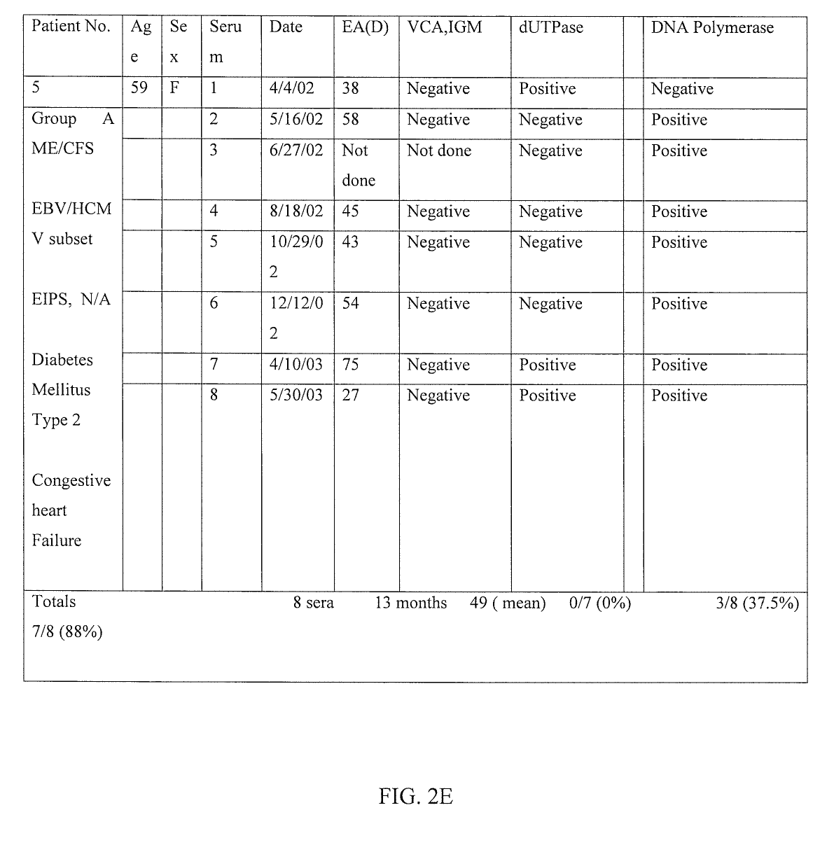

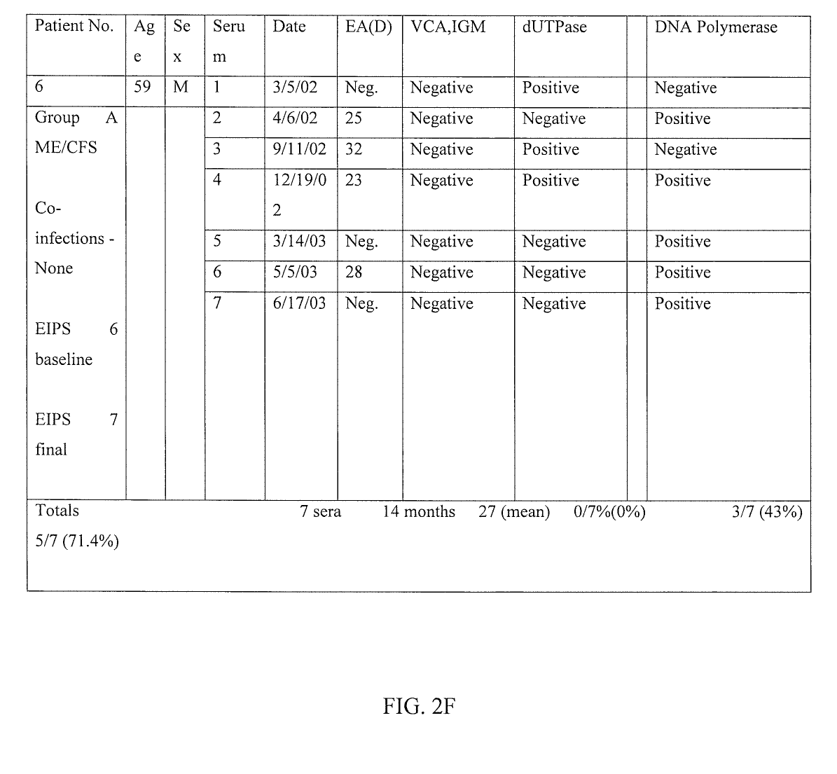

### ME/CFS Patients (FIGS. 2A-2F)

Demographics. ME/CFS patients (FIGS. 2A-2F) the six ME/CFS patients (5 women) were selected from 142 ME/CFS of the 2001-2007 systematic review. They were 37-59 years old. Serum samples were taken at intervals 3/5/02-11/14/03. There were 7 to 10 sera from each patient. Five of the 6 ME/CFS patients were Group A (no co-infections), and one patient had co-infection with Borrelia burgdorferi (patient no. 2). Five of the 6 patients were HCMV IgG serum antibody negative. Initial EIPS values were 3.5-5.0, meaning that patients could be out of bed only 3 to 4 hours a day, and required daily naps in bed to complete each day. One ME/CFS patient was able to struggle to complete a sedentary working day (FIG. 2F, patient no. 6). The solitary man of these 6 “ME/CFS” patients did not meet criteria for ME/CFS at baseline (EIPS, 6). He struggled at baseline to maintain his sedentary working day, required a daytime nap, and could no longer do any exercise without marked syncope and worsening fatigue. One year later the final EIPS values were 7-8, for the 5 group A ME/CFS patients, meaning that patients could now live normal lives. The single group B ME/CFS patient's final EIPS value increased from a baseline of 3.5 to 5, but this woman still met international criteria diagnosis of ME/CFS. The EIPS is a validated (FSS-9 item scale with high degree of internal consistency measured by Cronbach's alpha) is a Functional Activity Appraisal: energy Index Score Healthcare worker Appraisal.

EBV Encoded Gene Products EBV, VCA IgM Forty-nine VCA IgM assays were done. All were negative.

EBV, EA (D) Forty-nine EA (D) assays were done. All were positive except two sera from ME/CFS patient number 6 whose baseline EIPS value was 6. Mean patient EBV (EA) titers were: 54 (patient one); 123 (patient two); 63 (patient three); 128 (patient four); 49 (patient five); and 27 (patient six), negative <20. The mean EA (D) titer for these 6 patients was 74.

EBV dUTPase Three of 9 (33.3%), ME/CFS (patient one); 5 of 7 (71.4%) ME/CFS (patient two); 3 of 9 (33%) ME/CFS (patient three); 8 of 10 (80%) ME/CFS (patient four); 3 of 8 (37.5%) ME/CFS (patient five); 3 of 7 (43%) ME/CFS (patient six) were positive assays for elevated serum antibody titers to EBV dUTPase. Twenty-five of the 50 (50%) assays were positive.

EBV DNA polymerase. Eight of 10 (80%) of ME/CFS (patient one); 4 of 7 (57.1%) ME/CFS (patient two); 7 of 10 (70%) ME/CFS (patient three); 9 of 10 (90%) ME/CFS (patient four); 7 of 8 (88%) ME/CFS (patient five); and 3 of 5 (71.4%) (patient six) were positive serum assays for elevated antibody titers to EBV DNA polymerase. Forty of the 52 (76.9%) ME/CFS assays from ME/CFS patients were positive.

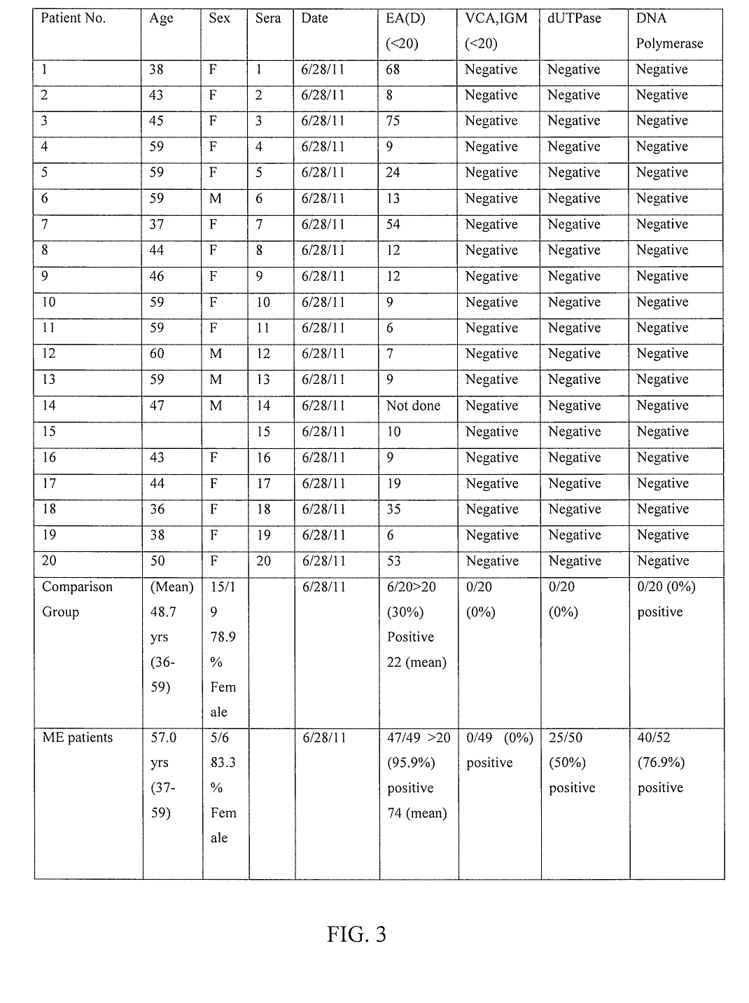

### Comparison Group Patients (FIG. 3)

### Demographics

The mean age of the comparison group was 48.7 years (36-59). Fifteen of 19 (78.9%) persons were women. EBV, VCA, IgM Twenty assays from the comparison group were done. All were negative.

EBV, EA (D) Twenty assays from the comparison group were done. Fourteen comparison group patients had negative EV EA (D) titers. Six comparison group patients had EBV (EA) titers. The mean EA (D) of the comparison group was 22.

EBV dUTPase twenty assays from the comparison group were done. All were negative.

EBV DNA polymerase twenty assays from the comparison group were done. All were negative.

### Analysis of FIGS. 2 and 3

These data demonstrate the presence of elevated serum antibodies to encoded EBV non-structural proteins DNA polymerase (EBV poly) and dUTPase (EBV dUTP) in blood from six EBV subset ME/CFS patients. These antibodies in the blood samples of ME/CFS patients accompany their likely source, a “primary” plasma cell apoptosis. EBV poly and dUTP-emias are being monitored in the ME/CFS patients. It is believed that EBV lytic virus had likely originated in pharyngeal epithelial cells to then infect adjacent memory B cells where the EBV genome was latent. As memory B cells differentiate to plasma cells, they are believed to be the probable site of the current early encoded EBV proteinemia abortive lytic replication. While the early EBV protein BZLF1 (Zta, EB1) initiates the EBV viral lytic cycle, only 15% of B cells expressing BZLF1 achieve a full lytic cycle to primary infection (infectious mononucleosis)—completing the virion. The majority replication is abortive lytic in type. It is believed that that the encoded circulating proteins other EBV encoded tegument early proteins may enter host cells of affected ME/CFS patient's organs, namely heart, striated muscle, and the brain to initiate a secondary expansive apoptosis along with IL-6, I-10, TGF-B, tyrosine Kinase, TKT matrix metalloproteinase and C-Fos. This secondary ME/CFS apoptosis is believed to be the pathologic mechanism of ME/CFS and does not require EBV DNA, and further may de-mystify and explain the difficulty in associating EBV replication and ME/CFS.

The proteins EBV dUTPase and EBV DNA polymerase separated the EBV subset ME/CFS patients from 20 comparison group patients, and, thus, helped narrow the field of possible molecular markers for the diagnosis of EBV subset ME/CFS patients. Assays for EBV, VCA IgM; EBV, EA(D); EBV dUTPase and EBV DNA polymerase along with Fukada/Carruthers ME/CFS criteria and the confirming diagnostic panel described define the EBV subset of ME/CFS patients. (FIG. 2) Five of the six ME/CFS patients at baseline met international criteria for ME/CF S. The single male was chronically ill, could not work regularly or exercise.

Forty-nine EBV VCA IgM serum assays taken serially over 13-16 months were negative. Comparison group assays for EBV VCA IgM, EBV dUTPase and EBV DNA polymerase were negative. However, 47/49 (95.9%) EBV EA (D) assays from ME/CFS patients were positive. Twenty-five of 50 (50%) dUTPase antibody serum assays were positive and 40/52 (76.9%) DNA polymerase serum antibody assays were positive from the ME/CFS patients. The results describe abortive lytic EBV replication of early viral proteins EA (D) dUTPase, and DNA polymerase which have been released from infected cells into the blood. These aberrant virus tegument-belonging, intracellular belonging proteins and, perhaps, other early EBV proteins may traverse cellular membranes of multiple host systems stimulating the immune dysregulation and the symptoms of ME/CF S. Serum antibodies of EBV dUTPase and EBV DNA polymerase in blood samples of ME/CFS patients, who have been treated or not treated with valacyclovir are present for over 400 days.

EBV dUTPase (BLLF3) is a part of this EBV early antigen complex. EBV DNA polymerase (BALF5) is an early protein heralding EBV lytic replication. EBV DUTPase catalyzes the hydrolysis of dUTP to dUMP, preventing incorporation of uracil into replicating DNA. EBV DNA polymerase is necessary for EBV lytic replication. EBV dUTPase and EBV Zta (BZLF1, 2EBRA, EB1) induce widespread immune dysregulation. Accordingly, abortive lytic EBV replication is believed to be responsible for the cardiomyopathy/encephalopathy of ME/CFS.

In another aspect, the invention provides methods of diagnosing an EBV subset of CFS patients, comprising using a serum diagnostic assay, the BMRF1 antigen, as a biomarker of EBV abortive lytic replication. In one embodiment, a method of diagnosing EBV subset of CFS includes the following protocol: obtaining from the patient a sample of biological or serological fluid in which the BMRF1 biomarkers would be found if the pathogen is present in the patient; measuring the biological sample for the presence of BMRF1 levels; and identifying the patient as having EBV abortive lytic replication if the identified BMRF1 biomarker is significantly above an established reference level. As used herein, a serologic or biologic level is significantly above the reference level if the serologic level exceeds the reference level to the degree that it would indicate the presence of an infection to a person of ordinary skill in the art. “Serologic marker” encompasses any serologic evidence that indicates the presence of the pathogen in the patient's body. Such evidence can include, for example, the presence of a molecule or other entity—such as the two molecular markers identified herein for the EBV subset of CFS patient—that is generally not present in the healthy individual; increase or decrease of the level of the molecule or other entity of what is generally present in healthy individual; or any other indicator know in the arts.

During the initial primary herpes virus infection, EBV antibodies to multiple early, middle and late gene products, as well as those to complete virus particles, are produced, which may give rise to serum-specific IgM antibody titers two multiple gene products. Then, as the patient recovers all serum antibody titers to early, middle and late herpes virus nonstructural genes disappear, any only positive serum antibody titers to complete structural virions, such as serum-specific IgG remain.

In CFS, however, there is “abortive” herpes virus infection with no complete virion multiplication, and products in the periphery may induce elevated serum antibody titers.

Therefore, in EBV subset CFS there may be elevated serum antibody titers to EBV early, middle and late gene products not ordinarily present. In group A CFS in which abortive replication by two or three of the EBV, HCMV, HHV6 is present, appropriate dual or triple herpes virus elevated early, middle, late serum antibody titers to gene products may be found.

### Assay Development and Optimization

The set of proteins necessary for replication of EBV, the EBV EA(D) replication complex, is composed of six viral proteins: the DNA polymerase BALF5, DNA polymerase processivity factor BMRF1, primase BSLF1, primase associated factor BBLF2/3, helicase BBLF4 and the single stranded DNA binding protection BALF2. All of the early encoded antigens were tested, as specified below. EBV early encoded antigens, BALF1, BLLF3a, BZLF1 and BMRF1, proteins were prepared from plasmids using E. coli or yeast.

DNA polymerase catalytic subunit, BALF5, string B-95-8, Human Herpesvirus 4, yeast derived, with an immunogen sequence length of 1015 with a purity of greater than 90% and 50% glycerol was tested.

EBV DUTPase, BLLF3a, deoxyuridine triphosphatase, yeast-derived with a mol. Wt 30 KD, pH of 7.4, 50% glycerol and an expression region 1-278 ua was tested.

EBV (BZLF1) EB1, Zebra Transactivator Protein, with an Immunogen Sequence 1-245, EBV B95-8, derived from yeast, with a pH 7.4 and 50% glycerol was tested.

BMRF1: DNA polymerase processivity factor EA-D polymerase accessory subunit, Immunogen sequence 404, derived from yeast, with a pH of 7.4 and 50% glycerol was tested.

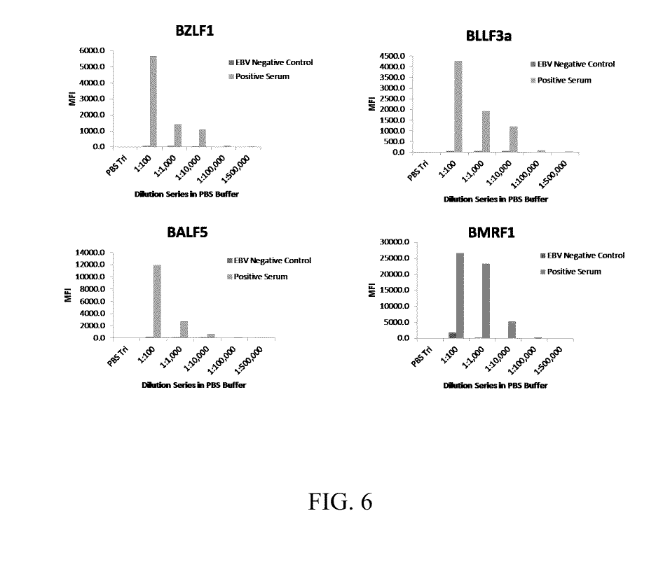

Four Epstein-Barr virus (EBV) recombinant antigens were selected by the CFS Foundation to test their utility as a diagnostic assay. These antigens were BMRF1, BZLF1, BLLF3 and BALF5 (Table 1). The antigens were conjugated to Luminex magnetic beads, with each antigen assigned a different bead region to allow for multiplexing, if more than one antigen was identified as EBV CFS specific. In addition, two different secondary antibodies were tested (a general anti-human antibody, and an anti-human IgM antibody). However, the goat anti-Human IgM, Fc5μ fragment specific antibody did not produce a signal differential in any of the four antigens tested (results not shown). The general anti-human antibody was therefore selected as the secondary antibody for the assay. The four antigen-bead conjugates were tested with known negative and positive human serum (against EBV CFS) for specificity. Specificity is defined as a signal differential between positive and negative sera. Positive and negative sera were serially diluted out in a ten-fold concentration range, and tested using the Luminex 200 instrument. Both positive and negative sera were provided by the CFS Foundation; the positive serum was pooled from twenty individuals; the negative serum was from one representative individual. Results from this study are shown in FIG. 6. Of the four antigens tested, only the BMRF1 antigen demonstrated a signal differential between the known positive and negative sera

TABLE 1
EBV ANTIGENS.
FOUR EBV ANTIGENS WERE SELECTED BY CFS FOUNDA-
TION, PRODUCED RECOMBINANTLY, AND CONJUGATED
TO BEADS FOR DIAGNOSTIC TESTING

| Bead | Immobilized | | |
|---|---|---|---|
| Region | on bead | Assay Detects | 2° Ab |
| 13 | BMRF1 | DNA polymerase proces- | Goat Anti-Human |
| | | sivity factor BMRF1 | IgA + IgG + IgM |
| 26 | Trans-activator | Trans-activator protein | (H + L) |
| | protein BZLF1 | BZLF1 | OR Goat Anti- |
| 39 | Recombinant | Epstein-Barr virus (strain | Human IgM, |
| | DUTPase | GD1) (Human herpesvirus | Fc5μ fragment |
| | (BLLF3) | 4 - HHV-4) | specific |
| 52 | BALF5 | DNA polymerase catalytic | |
| | | subunit (BALF5), partial | |

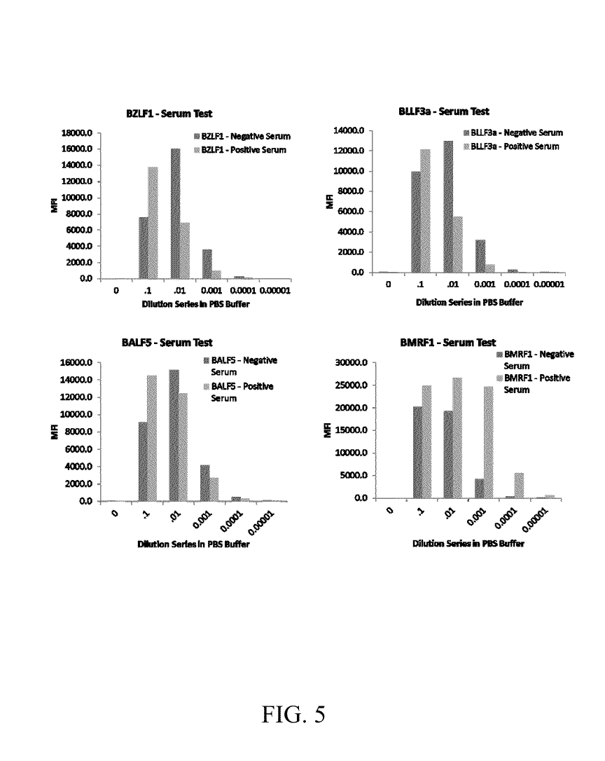

As observed in the FIG. 5 Reference Serum Test, the BMRF1 assay produces a signal differential between the negative and positive serum samples at all concentrations tested, whereas the other three antigens (BZLF1, BLLF3A and BALF5) do not. As observed in FIG. 6, no differentiation between positive (green) and negative (red) serum was observed for BZLF1, BLLF3, and BALF5, whereas nice signal differentiation was seen for BMRF1. The assays were then optimized to see if better resolution could be attained by changing the bead volume, adjusting reagent ratios, adding BSA, etc. However, no improvement was observed (data not shown).

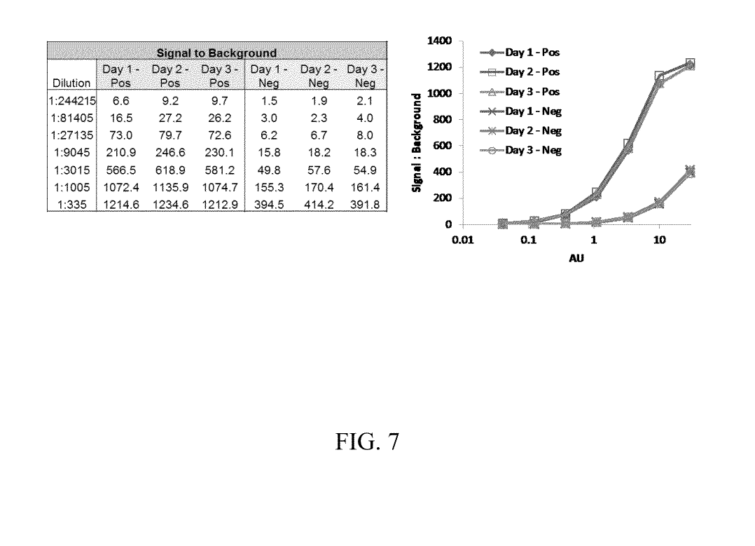

The four antigen-bead conjugates were then tested with a confirmed EBV negative control provided by CFS Foundation (FIG. 7). All four assays were negative when the confirmed EBV negative control was used, demonstrating that the assays are performing as expected. Therefore, it appears that the negative samples were cross-reactive (to a similar antigen, perhaps), limiting the diagnostic utility of the antigens.

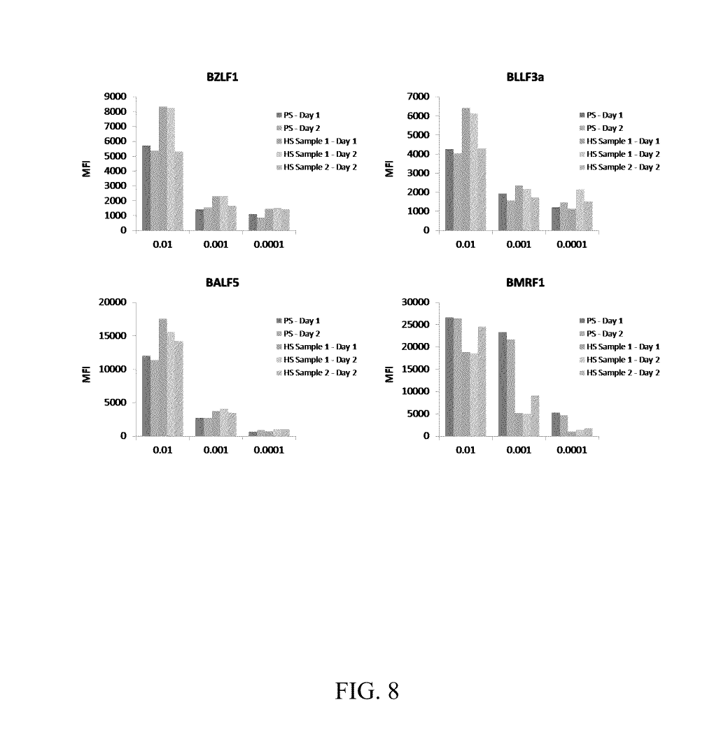

For confirmation, all four assays were tested with serum from healthy individuals. Similar to the negative test described above, a signal differential between the known EBV positive sample and the healthy individual(s) should be observed for the assay to perform as a diagnostic. Two healthy serum samples were tested: one sample that was pooled from ten healthy males (Sample 1), and one from a single individual, sex unknown (Sample 2). Sample 1 was run with the positive serum on two separate days, whereas Sample 2 was only tested on the second day. Results, as shown in FIG. 8, demonstrate that, similar to the positive and negative sera test, only the BMRF1 assay demonstrated a signal differential between healthy and known EBV positive individuals, supporting its further development as a diagnostic assay.

As shown in FIG. 7, BMRF1, but not the other antigens, is specific when compared with serum from healthy individuals, as observed by the signal differential between the pc and hs samples. PS—positive serum. HS—healthy serum. Sample 2 was only measured once, on day 2. In summary, for BZLF1, BLLF3a and BALF5 no signal differential was observed between a known EBV positive and the other samples tested (negative and healthy sera). Therefore, these antigens are not useful as a diagnostic test. In contrast, BMRF1 has a differential signal in every serum sample tested; therefore it appears to be specific for CFS. Data was reviewed by the sponsor and approve to move forward on Aug. 8, 2014 with BMFR1. Development proceeded with the BMRF1 assay as a diagnostic test for EBV CFS.

The next step in the assay development of BMRF1 was to establish the reference range for the standard curve. Positive and negative reference sera were run across a three-fold dilution range starting at 1:335 (the top of the linear range). This experiment was repeated three times to demonstrate reproducibility over the linear range of the assay. These sera samples were therefore established as the reference serum for the BMRF1 assay. A summary of the results is shown in FIG. 8. In FIG. 8, AU=Assay Units (1/Dilution Factor*10,000). These are random units used to fit the standard curve and interpolate data, if needed

Pooled Sera from Six Group a ME/CFS Patients: BMRF1 Positive

There were four women, and two men, mean age 40.3 years (range 23-57 years) who had been ill for a mean of 2.3 years at baseline (range 0.5-5 years). The mean BMI was 24.8 kg/m² (range 20-28). CMV IgG titers were negative in these six EBV subset patients. HHV6 mean IgG titers were negative in 2 patients: values were 5.2-14.9 in the others. The mean baseline EBV EA(D) titer was 66 (<20) (range 24-130).

Ten control ME/CFS patients had EBV EA(D) titers, which were negative. Their mean age was 46.8 years (range 15-84 years).

### BMRF1 Negative ME/CFS Patients (FIG. 3)

Six CMV titers among control patients were negative. Variable CMV (22-214_were present in 4 ME/CFS patients. HHV6 IgG titers were present in these 10 ME/CFS patients.

### Individual Sera of ME/CFS Patients EBV (EA) Positive, BMRF1 Positive (Table II)

Sera from the six ME/CFS patients with antibody to BMRF1 (Table 1) were obtained 1 month (two patients), 2 months (one patient), 6 months (one patient), 12 months (one patient) and 24 months (one patient) after baseline.

### Control SEID Sera Negative to EBV EA(D)

A single control serum from a healthy 34-year-old woman negative to EBV EA(D), CMV, and HHV6 was used. Ten individual sera from patients with SEID of unknown cause were studied. Each of these SEID patients had no serum antibody to EBV EA(D). It is believed that these SEID patients belong to Group A HHV6/CMV SEID subsets.

Control Random Non-Identified Patients from a Diagnostic Laboratory in California

Nineteen random patients whose only identification was their age and sex had BMRF1 Luminex assays were performed. Similar assays were performed on 26 additional EBV subset patients.

TABLE 2
SAMPLE TYPE AND NUMBER.

| | | | Parameter |
|---|---|---|---|
| Sample type | Sample number | Sample Description | addressed |
| Positive | 6 patient samples | EBV seropositive | Sensitivity, |
| | | samples | Precision |
| Negative | 10 patient samples | EBV seronegative | Specificity, |
| | | samples | Precision |

### Results

The BMFR1 positive Luminex Platform produced a signal between the negative and positive serum samples at all concentrations tested. At all concentrations tested the antigens BZLF1, BLLF3 and BALF were negative. Positive and negative tests are shown, FIG. 1. The standard curve is shown, FIG. 2. The BMRF1 individual results are shown, Table IV. Sensitivity, quantization, precision, reproducibility was present. It is understood that EBV DNA processivity factor, BMRF1, which is intimately complexed with EBV DNA polymerase, and BMRF1's function is necessary for EBV DNAchain lengthening is the identifying biomarker for EBV subset ME/CFS patients.

Results demonstrate acceptable sensitivity, as there is a clear distinction between EBV seronegative and EBV seropositive samples at 1:3,000 (Table 4). One sample, ID 9779, tested as seronegative in the previous test, but was positive in the Luminex assay. This sample was the lowest positive sample observed, and the most likely explanation is therefore an increased sensitivity in the Luminex assay versus the previous test (this sample was below threshold previously but is above the threshold in the new assay). Results were also reproducible over the three days tested. Intra-plate precision was quite good, as it was generally under 5% for most samples tested. Inter-plate precision showed more variability, but was acceptable (under 15%) for most samples (the high and low samples had higher variability).

A summary of all results is shown in Table 4. Fifteen out of the sixteen samples tested behaved as expected; the exception was sample 9779, which was positive in our assay but tested negative at the CFS Foundation. As this was the lowest positive sample in the dataset, this discrepancy is likely due to a difference in sensitivity between the Luminex assay and the assay previously used to characterize the samples; sample 9779 was below the detection threshold in the previous analysis but above the cutoff in the current assay. These results demonstrate that the assay is both sensitive (results can be determined at even at very low concentrations—1:250,000) and specific with a clear distinction between negative and positive sample results).

TABLE 3
SUMMARY OF ALL RESULTS.

| | | | Signal:Background | | | | Interpolated | | |
|---|---|---|---|---|---|---|---|---|---|
| | | | 1:3,000 | | 1:250,000 | | 1:3,000 | | |
| | | | No. above | Mean | No. above | Mean | No. above | Mean | |
| | Sample | | cutoff/ | (Cutoff = | cutoff/ | (Cutoff = | cutoff/ | (Cutoff = | |
| Number | Type | Sample ID | Total | 100) | Total | 2.5) | Total | 1.5) | Result |
| Sample 1 | Positive | ID: 11212 | 12/12 | 407.18 | 12/12 | 7.11 | 12/12 | 10.09 | Positive |
| Sample 2 | Negative | ID: 11223 | 0/12 | 52.46 | 0/12 | 1.79 | 0/12 | 0.92 | Negative |
| Sample 3 | Negative | ID: 1100684 | 0/12 | 26.38 | 0/12 | 1.45 | 0/12 | 0.38 | Negative |
| Sample 4 | Positive | ID: 11391 | 12/12 | 195.74 | 12/12 | 3.61 | 12/12 | 4.08 | Positive |
| Sample 5 | Positive | ID: 11351 | 12/12 | 273.41 | 12/12 | 4.67 | 12/12 | 6.04 | Positive |
| Sample 6 | Negative | ID: 10516 | 0/12 | 14.86 | 0/12 | 1.26 | 0/12 | 0.16 | Negative |
| Sample 7 | Negative | ID: 11385 | 0/12 | 43.23 | 0/12 | 2.00 | 0/12 | 0.47 | Negative |
| Sample 8 | Negative | ID: 9779 | 12/12 | 149.20 | 9/12 | 2.89 | 12/12 | 2.54 | Positive |
| Sample 9 | Negative | ID: 11163 | 0/12 | 64.51 | 1/12 | 2.14 | 0/12 | 0.87 | Negative |
| Sample 10 | Positive | ID: 11191 | 12/12 | 376.36 | 12/12 | 5.79 | 12/12 | 7.27 | Positive |
| Sample 11 | Negative | ID: 11437 | 0/12 | 34.17 | 0/12 | 1.67 | 0/12 | 0.35 | Negative |
| Sample 12 | Negative | ID: 11077 | 0/12 | 36.29 | 0/12 | 1.53 | 0/12 | 0.35 | Negative |
| Sample 13 | Negative | ID: 11333 | 0/12 | 53.04 | 0/12 | 1.88 | 0/12 | 0.93 | Negative |
| Sample 14 | Positive | ID: 11170 | 12/12 | 343.04 | 12/12 | 5.39 | 12/12 | 6.16 | Positive |
| Sample 15 | Negative | ID: 11381 | 0/12 | 28.03 | 0/12 | 1.61 | 0/12 | 0.52 | Negative |
| Sample 16 | Positive | ID: 11178 | 12/12 | 668.23 | 12/12 | 13.92 | 12/12 | 14.14 | Positive |

FIG. 5. EBV ANTIGEN NEGATIVE CONTROL TEST

All assays are negative when a confirmed EBV negative control is used.

FIG. 8. STANDARD CURVE

These are random units used to fit the standard curve and interpolate data, if needed. AU=Assay Units (1/Dilution Factor*10,000).

### BMRF1 Luminex Assays (Table 4, FIG. 4)

Twenty-six EBV subset, EA(D) positive SEID patients (mean age 46.2, 69% female) were assayed. All 26 EBV subset patients had elevated serum BMRF1 titers (mean 19.7). The 19 unidentified random control patients (mean age 37.4, 69% female) from the diagnostic laboratory had a mean BMRF1 value, 2.2. For age and BMRF1, t-tests were used to examine differences. Gender, chi-square analysis was used. No significant statistical difference was indicated for % women and age between the SEID patients and the control group. A significant difference was indicated for BMRF1, the SEID patients had much higher values, p<0.001. We suspect the unidentified random control patient was EBV subset SEID.

TABLE 4
Comparison between EBV subset SEID
patients and the control group

| | SEID Patients | | Control Group | | |
|---|---|---|---|---|---|
| | % | (n) | % | (n) | p* |
| Percent | 69 | (n = 26) | 69 | (n = 13) | 1.00 |
| Women | | | | | |
| | Mean ± SD | (n) | Mean ± SD | (n)) | |
| Age (Years) | 46.2 ± 13.7 | (n = 26) | 37.4 ± 18.9 | (n = 14) | 0.10 |
| BMRF1 | 19.7 ± 18.6 | (n = 26) | 2.2 ± 3.7 | (n = 19) | <0.001 |

*Gender distributions were compared by chi Square analysis, age and BMRF1 were compared by t-tests.
Mean BMRF1 serum antibody titers in EBV subset SEID patients and controls. Mean BMRF1 Luminex assay serum antibody titers in 26 EBV subset SEID patients and 19 random controls.

### Conclusions

The hypothesis that EBV subset ME/CFS patients may be identified by the presence of serum early-encoded proteins was tested, which may also be a pathologic mechanism in EBV abortive lytic replication. We used four EBV Early Encoded purified protein antigens: BALF5 (DNA polymerase); BLLF3a (deoxyuridine triphosphatase); BZLF1 (EB1, Zebra, transactivator protein) and BMRF1 (DNA polymerase processivity factor). We previously reported that neutralization test assays of Early Encoded antibody to EBV DNA polymerase (BALF5) and EBV dUTPase (BLLF3) may identify EBV subset ME/CFS.[10] Here, Luminex technology antibody to BALF5, BZLF1 and BLLF3 were negative. Antibody to BMRF1, the DNA polymerase accessory factor identified EBV abortive lytic replication in EBV subset ME/CFS patients.[7]

The Luminex assay for serum antibody to BMRF1 is a diagnostic biomarker for EBV subset ME/CFS.

These data indicate that EBV subset SEID patients may be distinguished by an elevated IgG serum BMRF1 antibody which persisted for at least 12 months. BMRF1 is an EBV early-encoded protein, the DNA polymerase accessory processivity factor which must complex with BALF5, DNA polymerase to facilitate viral chain lengthening permitting lytic replication.[18,21,22] During EBV lytic replication BMRF1 is intranuclear either within affected B-lymphocytes or within affected epithelial cells in the tonsils. [26-28] BMRF1 is one of six EBV Early Encoded proteins.[18] Antibody to this Early Encoded EBV protein complex may be present “transiently” in primary infection, including infectious mononucleosis (IM). However, within 6 months after IM, this EA complex antibody is absent in patients who have recovered from this lytic infection.

BMRF1 antibody may define EBV abortive lytic replication, and is a diagnostic biomarker for EBV subset SEID disease. Here elevated BMRF1 serum antibody was present and persistent for at least 12 months in 6 EBV subset SEID patients whom we followed. BMRF1 antibody was absent in 10 SEID patients suspected to have CMV or HHV6 subset SEID, but not EBV subset SEID. EBV BMRF1 (mean titer 19.7±18.6) was present in 26 EBV subset SEID patients and absent in random controls (n=19), BMRF1 mean titer 2.2±3.7. BMRF1 as well as other aberrant Early Encoded proteins in the periphery may be responsible candidates for abnormal brain magnetic resonance imaging, abnormal cytokines; abnormal myocardial function; anticardiac antibodies (including antibody to the myocardial cytoskeletal desmin), and the other functional abnormalities we provisionally named Systemic Exertion Intolerance Disease, SEID.

Using a Luminex antibody BMRF1 gene-specific assay, we associate this herpesvirus DNA polymerase accessory processivity factor with EBV subset SEID. Earlier, we associated EBV subset SEID with antibody to DNA polymerase BALF5 and/or dUTPase (BLLF3) by neutralization tests. We may have assayed BMRF1 antibody, in an unrecognized antigen complex, EBV DNA polymerase-EBV DNA polymerase accessory factor. We also earlier reported elevated serum antibody to the Early Encoded proteins CMV p52 (UL44) and CM₂ (UL44 complexed with UL57) in patients with CMV subset SEID. CMV UL44 p52 is the CMV DNA polymerase accessory processivity factor, exactly the same critical gene function in CMV replication, as that of EBV BMRF1, the latter gene the purpose of this present report.[34,35] In this regard Rasmussen, Draborg, and Nielson, et al recently reported that in some patients with systemic lupus erythematosus there are elevated IgG serum antibody titers to EBV BMRF1 and CMV p52, both of which are DNA polymerase accessory factors for these respective herpesviruses. Certainly, any immunologic stressor, theoretically, may activate herpesvirus abortive lytic replication. Further research is required to substantiate this hypothesis.

Multiple preliminary and larger systemic reviews of antiviral therapy of “purported” EBV, CMV and Human Herpesvirus 6 subset Group A SEID patients have been reported by our group and have been therapeutically successful in patients with SEID. Antiviral nucleosides: valacyclovir, valganciclovir, famciclovir or the nucleotide cidofovir (unpublished studies) all have had salutary results in SEID patients. These antiviral agents inhibit the respective DNA polymerases of the appropriate CMV, EBV, HHV6 herpesviruses.[8] A placebo controlled trial of these antiviral agents using the appropriate diagnostic biomarkers we suggest supports a well organized large-enough blinded placebo-controlled trial of therapy. Herpesvirus (EBV, CMV, HHV6) abortive lytic replication may be associated with SEID.

### Assay Development and Optimization

To specifically diagnose ME/CFS, a serological assay was prepared based on EBV viral products specific to the ME/CFS diagnosis. To develop the diagnostic assay, a bead-based fluorescence detection instrument was used which operates on the principles of flow cytometry. The immunoassays were developed on a Luminex™ 200 instrument using microsphere immunofluorescent bead-based technology capable of simultaneously analyzing a single specimen for multiple analytes. Additionally, the beads chosen for use in the assay have downstream capability with other systems, such as the MAGPIX™, introduced by Luminex, which uses magnetic microspheres.

Analytical validation are preferably performed for each antigen in a singleplex assay, and then all together in a multiplex assay. Preferably, analytical sensitivity and specificity within the detection limit are performed for each antigen individually in a singleplex assay and then combined in a multiplex assay.

Luminex Technology Platform.

Luminex's xMAP technology, based upon flow cytometry, color-codes tiny beads, called microspheres of various fluorescent dyes yielding different color regions, into up to 100 distinct sets. Each bead set can be coated with a reagent specific to a particular bioassay (antigens), in this case the EBV antigen, allowing the capture and detection of specific analytes from a sample. The microspheres are magnetically charged and the internal dyes are excited by a laser, marking the microsphere set. A second laser excites the fluorescent dye on the reporter molecule. Finally, high-speed digital-signal processors identify each individual microsphere and quantify the results of its assay, based on fluorescent reporter signals.

### Selection of Antigens Based on EBV Viral Genes

Four EBV recombinant antigens were selected to test their utility as a diagnostic assay. These antigens were BMRF1, BZLF1, BLLF3 and BALF5 (FIG. 5). The antigens were conjugated to Luminex magnetic beads, with each antigen assigned a different bead region to allow for multiplexing, if more than one antigen was identified as EBV CFS specific. In addition, two different secondary antibodies were tested (a general anti-human antibody, and an anti-human IgM antibody). However, the goat anti-Human IgM, Fc5μ fragment specific antibody did not produce a signal differential in any of the four antigens tested. The general anti-human antibody was therefore selected as the secondary antibody for the assay. This antibody was biotinylated prior to use in the assay, and detection was via streptavidin-phycoerythin (SAPE) binding to the biotinylated secondary antibody. To perform the Luminex assay, samples were diluted in PBS and loaded onto a round bottom 96 well plate. Bead/capture antigen mixture was diluted with PBS-TBN (PBS plus 0.1% BSA, 0.02% Tween-20 and 0.05% Sodium Azide) and loaded onto the plate as well. The plate was then incubated for 1 hour, shaking at room temperature. The plate was washed three times with PBS-TBN, secondary antibody was added, and incubated for 30 min, shaking at room temperature, protecting plate from light. The plate was washed three times again and SAPE was added. Then the plate was incubated for 30 min, shaking at room temperature, while protecting from light. The plate was then loaded onto the Luminex 200 instrument for analysis. To compare the four EBV antigen assays, raw MFI values were first normalized to blank (PBS only) values, in order to account for any difference in background. For analysis of patient samples, raw median fluorescent intensity (MFI) values from the standard curve were fitted using a four parameter nonlinear regression model. Sample MFI data were interpolated to this curve. Data are expressed as assay units (AU), where AU equals (1/dilution factor)/*10.
Twenty-six additional EBV subset SEID patients and 19 random control patients from a diagnostic laboratory in California were assayed for serum BMRF1 antibody titers using Luminex technology.

The four antigen-bead conjugates were tested with known negative and positive human serum (against EBV CFS) for specificity.

Specificity is defined as a signal differential between positive and negative sera. Positive and negative sera were serially diluted out in a ten-fold concentration range, and tested using the Luminex 200 instrument. Both positive and negative sera were provided by the CFS Foundation; the positive serum was pooled from twenty individuals; the negative serum was from one representative individual. Of the four antigens tested, only the BMRF1 antigen demonstrated a signal differential between the known positive and negative sera.

TABLE 5
EBV ANTIGENS.
FOUR EBV ANTIGENS WERE SELECTED BY CFS FOUNDA-
TION, PRODUCED RECOMBINANTLY, AND CONJUGATED
TO BEADS FOR DIAGNOSTIC TESTING

| Bead | Immobilized | | |
|---|---|---|---|
| Region | on bead | Assay Detects | 2° Ab |
| 13 | BMRF1 | DNA polymerase proces- | Goat Anti-Human |
| | | sivity factor BMRF1 | IgA + IgG + IgM |
| 26 | Trans-activator | Trans-activator protein | (H + L) |
| | protein BZLF1 | BZLF1 | OR Goat Anti- |
| 39 | Recombinant | Epstein-Barr virus (strain | Human IgM, |
| | DUTPase | GD1) (Human herpesvirus | Fc5μ fragment |
| | (BLLF3) | 4 - HHV-4) | specific |
| 52 | BALF5 | DNA polymerase catalytic | |
| | | subunit (BALF5), partial | |

No differentiation between positive and negative serum was observed for BZLF1, BLLF3, and BALF5, whereas nice signal differentiation was seen for BMRF1. The assays were then optimized to see if better resolution could be attained by changing the bead volume, adjusting reagent ratios, adding BSA, etc. However, no improvement was observed.

The four antigen-bead conjugates were then tested with a confirmed EBV negative control provided by the CFS Foundation. All four assays were negative when the confirmed EBV negative control was used, demonstrating that the assays are performing as expected. Therefore, it appears that the negative samples were cross-reactive (to a similar antigen, perhaps), limiting the diagnostic utility of the antigens.

For confirmation, all four assays were tested with serum from healthy individuals. Similar to the negative test described above, a signal differential between the known EBV positive sample and the healthy individual(s) should be observed for the assay to perform as a diagnostic tool. Two healthy serum samples were tested: one sample that was pooled from ten healthy males (Sample 1), and one from a single individual, sex unknown (Sample 2). Sample 1 was run with the positive serum on two separate days, whereas Sample 2 was only tested on the second day. Results demonstrate that, similar to the positive and negative sera test, only the BMRF1 assay demonstrated a signal differential between healthy and known EBV positive individuals, supporting its further development as a diagnostic assay.

In summary, for BZLF1, BLLF3a and BALF5 no signal differential was observed between a known EBV positive and the other samples tested (negative and healthy sera). Therefore, these antigens are not useful as a diagnostic test. In contrast, BMRF1 has a differential signal in every serum sample tested; therefore it appears to be specific for CFS.

The next step in the assay development of BMRF1 was to establish the reference range for the standard curve. Positive and negative reference sera were run across a three-fold dilution range starting at 1:335 to 1:244215. This experiment was repeated three times to demonstrate reproducibility over the linear range of the assay. These sera samples were therefore established as the reference serum for the BMRF1 assay.

### Analytical Validation of the Assay

The BMRF1 assay was then analytically verified using patient samples. The Test Design is shown in Tables 5 and 6. Six positive and ten negative sera were provided by the CFS Foundation. These samples were run in quadruplicate on three separate days. Two dilutions were used: 1:3,000 (which is in the middle of the linear range of the assay); and 1:250,000 (at the lower end of the concentration range of the standard curve). Samples received were blinded to the analyst prior to verification testing. The parameters analyzed were sensitivity, specificity, and precision.

TABLE 6
Sample type and number and test design.

| Sample type | Sample number | Sample Description | Parameter addressed |
|---|---|---|---|
| Positive | 6 patient samples | EBV seropositive samples | Sensitivity, Precision |
| Negative | 10 patient samples | EBV seronegative samples | Specificity, Precision |

| Day | Plate | Samples | Dilutions | Replicates | Sample Number | Total |
|---|---|---|---|---|---|---|
| 1 | 1 | 6 (3 Positive & | 1:3,000 | 4 | 48 (24 of each | 128 |
| | | 3 Negative) | 1:250,000 | | dilution) | |
| | 2 | 6 (1 Positive & | 1:3,000 | 4 | 48 (24 of each | |
| | | 5 Negative) | 1:250,000 | | dilution) | |
| | 3 | 4 (2 Positive & | 1:3,000 | 4 | 32 (16 of each | |
| | | 2 Negative) | 1:250,000 | | dilution) | |
| 2 | 1 | 6 (3 Positive & | 1:3,000 | 4 | 48 (24 of each | 128 |
| | | 3 Negative) | 1:250,000 | | dilution) | |
| | 2 | 6 (2 Positive & | 1:3,000 | 4 | 48 (24 of each | |
| | | 4 Negative) | 1:250,000 | | dilution) | |
| | 3 | 4 (2 Positive & | 1:3,000 | 4 | 32 (16 of each | |
| | | 2 Negative) | 1:250,000 | | dilution) | |
| 3 | 1 | 6 (3 Positive & | 1:3,000 | 4 | 48 (24 of each | 128 |
| | | 3 Negative) | 1:250,000 | | dilution) | |
| | 2 | 6 (2 Positive & | 1:3,000 | 4 | 48 (24 of each | |
| | | 4 Negative) | 1:250,000 | | dilution) | |
| | 3 | 4 (2 Positive & | 1:3,000 | 4 | 32 (16 of each | |
| | | 2 Negative) | 1:250,000 | | dilution) | |
| TOTAL | 9 plates | 16 samples | 2 | 4 of each | 168 positive and 216 | 384 total |
| | | | dilutions | dilution | negative assays | assays |

Although the data was not normalized and day-to-day variations not accounted for, the raw data data results showed a difference between positive and negative samples in both the 1:3,000 and 1:250,000 dilutions.

Once the data was normalized to the standard curve, the standard curve from each plate was plotted and fit using a four parameter nonlinear regression analysis. Sample data were then interpolated to the corresponding curve. Only the 1:3,000 was normalized to the standard curve, as the 1:250,000 concentration is outside of the linear range of the assay, so the result would not be accurate. Data normalized both ways look similar, with a clear distinction between positive and negative samples observed at the 1:3,000 dilutions. The distinction is still evident at the 1:250,000 dilution, demonstrating that the Luminex assay is sensitive at even very low levels, but the distinction is less defined. A cutoff for each normalized data set is also shown, in a dotted gray line. The cutoff was calculated by drawing a best fit line between the positive and negative datasets to define the assay threshold. Although it fits the current dataset, it may need to be adjusted as more patient samples are analyzed. Finally, as the 1:3,000 dilution performed better than 1:250,000 and normalization to background is simpler than normalizing to the standard curve, we recommend running the 1:3,000 dilution and normalizing to the background in the final diagnostic assay.

A summary of all results is shown in Table 7. Fifteen out of the sixteen samples tested behaved as expected; the exception was sample 9779, which was positive in our assay but tested negative at the CFS Foundation. As this was the lowest positive sample in the dataset, this discrepancy is likely due to a difference in sensitivity between the Luminex assay and the assay previously used to characterize the samples; sample 9779 was below the detection threshold in the previous analysis but above the cutoff in the current assay. These results demonstrate that the assay is both sensitive (results can be determined at even at very low concentrations—1:250,000) and specific with a clear distinction between negative and positive sample results).

TABLE 7
Summary of all results.

| | | | Signal:Background | | | | Interpolated | | |
|---|---|---|---|---|---|---|---|---|---|
| | | | 1:3,000 | | 1:250,000 | | 1:3,000 | | |
| | | | No. above | Mean | No. above | Mean | No. above | Mean | |
| | Sample | | cutoff/ | (Cutoff = | cutoff/ | (Cutoff = | cutoff/ | (Cutoff = | |
| Number | Type | Sample ID | Total | 100) | Total | 2.5) | Total | 1.5) | Result |
| Sample 1 | Positive | ID: 11212 | 12/12 | 407.18 | 12/12 | 7.11 | 12/12 | 10.09 | Positive |
| Sample 2 | Negative | ID: 11223 | 0/12 | 52.46 | 0/12 | 1.79 | 0/12 | 0.92 | Negative |
| Sample 3 | Negative | ID: 1100684 | 0/12 | 26.38 | 0/12 | 1.45 | 0/12 | 0.38 | Negative |
| Sample 4 | Positive | ID: 11391 | 12/12 | 195.74 | 12/12 | 3.61 | 12/12 | 4.08 | Positive |
| Sample 5 | Positive | ID: 11351 | 12/12 | 273.41 | 12/12 | 4.67 | 12/12 | 6.04 | Positive |
| Sample 6 | Negative | ID: 10516 | 0/12 | 14.86 | 0/12 | 1.26 | 0/12 | 0.16 | Negative |
| Sample 7 | Negative | ID: 11385 | 0/12 | 43.23 | 0/12 | 2.00 | 0/12 | 0.47 | Negative |
| Sample 8 | Negative | ID: 9779 | 12/12 | 149.20 | 9/12 | 2.89 | 12/12 | 2.54 | Positive |
| Sample 9 | Negative | ID: 11163 | 0/12 | 64.51 | 1/12 | 2.14 | 0/12 | 0.87 | Negative |
| Sample 10 | Positive | ID: 11191 | 12/12 | 376.36 | 12/12 | 5.79 | 12/12 | 7.27 | Positive |
| Sample 11 | Negative | ID: 11437 | 0/12 | 34.17 | 0/12 | 1.67 | 0/12 | 0.35 | Negative |
| Sample 12 | Negative | ID: 11077 | 0/12 | 36.29 | 0/12 | 1.53 | 0/12 | 0.35 | Negative |
| Sample 13 | Negative | ID: 11333 | 0/12 | 53.04 | 0/12 | 1.88 | 0/12 | 0.93 | Negative |
| Sample 14 | Positive | ID: 11170 | 12/12 | 343.04 | 12/12 | 5.39 | 12/12 | 6.16 | Positive |
| Sample 15 | Negative | ID: 11381 | 0/12 | 28.03 | 0/12 | 1.61 | 0/12 | 0.52 | Negative |
| Sample 16 | Positive | ID: 11178 | 12/12 | 668.23 | 12/12 | 13.92 | 12/12 | 14.14 | Positive |

Finally, to complete Task 1.2 assay precision was analyzed. Precision is calculated as the relative standard deviation (RSDs); RSD is calculated as the standard deviation of the replications divided by the mean of the replications, with a lower percentage indicating better assay precision. Generally, a RSD of ≦10% is considered ideal for an assay. Data normalized to background was used for this calculation, as this measure can be used for all samples and dilutions tested. Additionally, as normalized data will be used diagnostically, evaluating the precision of normalized samples is the most relevant dataset to analyze. RSD was calculated for the 1:3,000 dilution only, as this concentration is in the linear range and will be used in the final diagnostic assay. Both intra-plate (the four replicates run within the same plate) and inter-plate (all replicates) precision was calculated. Inter-plate precision was calculated two ways: (1) using all 12 replicates (all replicate set) and (2) by eliminating the highest and lowest replicate of each set and then using the middle ten replicates (adjusted set). This was a way to remove any potential outliers in the replicate set. Overall, the assay performed well, considering the limited number of replicates and plates run. Most samples had RSD values below 10% for both inter- and intra-plate precision; although intra-plate precision was better than inter-plate precision, as demonstrated by the number of samples with RSD values below 10% (Table 5). For intra-plate precision, only two samples (both on Day 1) had RSD values above 10%; all others were below. These were samples 11391 (11.8%) and 11178 (23.9%—this is likely due to a single outlier). For inter-plate precision, most samples were below 10%. When all 12 replicates were analyzed, 11/16 samples were below 10%, with the highest RSD value at 17.6% for sample 11391. For the adjusted replicate set (middle ten replicates), 12/16 samples were below 10%. Two of those were just above 10% (at 10.3% and 10.9%); the other two were at 15.3% and 15.6%. A further reduction in RSD values would be expected if more replicates were run.

TABLE 8
Precision.
Precision is calculated using relative standard deviation (RSD). A lower percent means
better precision. Precision was calculated using data normalized to background.

| | | | Intra-plate | | | Inter-plate | |
|---|---|---|---|---|---|---|---|
| | Sample | | 1:3,000 | | | 1:3,000 | |
| Number | Type | Sample ID | Day 1 | Day 2 | Day 3 | All | Adjusted* |
| Sample 1 | Positive | ID: 11212 | 1.7% | 1.0% | 1.3% | 2.9% | 2.5% |
| Sample 2 | Negative | ID: 11223 | 0.8% | 3.1% | 2.2% | 6.3% | 5.4% |
| Sample 3 | Negative | ID: 1100684 | 6.3% | 3.8% | 3.7% | 7.0% | 5.1% |
| Sample 4 | Positive | ID: 11391 | 11.8% | 2.9% | 2.5% | 17.6% | 15.3% |
| Sample 5 | Positive | ID: 11351 | 0.9% | 1.2% | 1.8% | 9.5% | 8.7% |
| Sample 6 | Negative | ID: 10516 | 4.6% | 2.1% | 1.7% | 16.2% | 15.6% |
| Sample 7 | Negative | ID: 11385 | 0.8% | 4.9% | 3.6% | 3.9% | 3.2% |
| Sample 8 | Negative | ID: 9779 | 2.1% | 3.4% | 3.5% | 11.4% | 10.3% |
| Sample 9 | Negative | ID: 11163 | 1.7% | 3.6% | 2.1% | 4.2% | 3.4% |
| Sample 10 | Positive | ID: 11191 | 1.0% | 1.3% | 1.8% | 8.9% | 8.1% |
| Sample 11 | Negative | ID: 11437 | 3.0% | 1.9% | 3.6% | 12.4% | 10.9% |
| Sample 12 | Negative | ID: 11077 | 1.4% | 7.2% | 1.3% | 8.5% | 7.4% |
| Sample 13 | Negative | ID: 11333 | 2.7% | 0.9% | 1.1% | 5.8% | 4.9% |
| Sample 14 | Positive | ID: 11170 | 2.3% | 3.0% | 1.6% | 2.3% | 1.9% |
| Sample 15 | Negative | ID: 11381 | 7.0% | 2.2% | 4.1% | 5.9% | 4.0% |
| Sample 16 | Positive | ID: 11178 | 23.9% | 3.2% | 2.6% | 15.8% | 6.5% |

| | | | | | |
|---|---|---|---|---|---|
| No. Samples RSD ≦10%/Total | 14/16 | 16/16 | 16/16 | 11/16 | 12/16 |
| No. replicates analyzed (per sample) | 4 | 4 | 4 | 12 | 10 |

*Adjusted samples were calculated by removing the highest and lowest value from each sample and then calculating the RSD.

### SUMMARY

We have developed a diagnostic point of care assay that can accurately stage EBV infection and enable accurate diagnosis of Group A myalgic encephalomyelitis/chronic fatigue syndrome (ME/CFS) for effective treatment. To accomplish this, we performed the following tasks:

We tested four candidate non-structural viral gene products from Epstein-Barr virus (EBV). One of these, BMRF1, was demonstrated to be specific to EBV seropositive individuals. It was further developed as a serum antibody assay to be used for early diagnosis of Epstein-Barr virus (EBV) subset ME/CFS.
Performance of this assay was verified using sixteen patient samples, six of which were known EBV positives and ten of which were negative (as determined previously by CFS Foundation using a different assay). The Luminex assay called fifteen of the sixteen samples as anticipated. The exception was sample 9779, which was positive in our assay, but negative according to previous results. As this was the lowest positive sample, the most likely explanation is that the Luminex assay is more sensitive; therefore sample 9779 is above the cutoff in the Luminex assay, but below the cutoff in the previous assay. Acceptable assay specificity, sensitivity, and precision were demonstrated. Furthermore, cutoff criteria to determine positive and negative samples were determined using this dataset.

Analytical validation will be performed for each antigen in a singleplex assay, and then all together in a multiplex assay. Analytical sensitivity and specificity with limit of detection will be performed for each antigen individually in a singleplex assay and then all together in a multiplex assay. Different concentrations of each antigen will be used for sensitivity and specificity studies ranging from the lowest detection limit through the highest detection limit. In addition, reproducibility, repeatability and precision studies will be performed using negative, low positive and high positive controls by testing these controls in multiple repeats within the run, between the run, over different days, different lots and different operators. Mean, standard deviation and CV will be calculated. Optimization will be performed until reproducibility and precision indicates a CV % less than 10. Studies will be compared with the existing EBV ELISA assay performed in the laboratory (Diasorin, Inc). After the optimization of each antigen, all of the antigens together will be compared with ELISA testing. Internal controls and internal calibrations will be also optimized in comparison to the ELISA assay. In addition other conditions, such as incubation time, wash solution and sample dilution as well as storage conditions will be optimized.

Reference ranges. Reference ranges will be established through base comparison with ELISA testing. Negative specimens will be measured by ELISA and then by Luminex multiplex assay to establish reference ranges. In addition positive patient specimens will be diluted to non-detection levels to confirm established reference ranges.

### Result Interpretation

In the IgG serology, EBV EA must be positive and should validate each reaction/assay. The control attached to bead #1 must be always negative, control attached to bead #10 must be weak negative, control attached to number #15 must be positive and control attached to #20 must be strongly positive. After all controls are positive, antibodies to either of the two new antigens: EBV encoded dUTPase and the EBV DNA encoded polymerase gene products must be positive for the test to be positive.

### Analytical Sensitivity

Analytical sensitivity will be studied in two ways: first, positive results will be diluted to the lowest detection levels and compared to ELISA assays, and second, pooled negative specimens will be spiked with known concentration of antibodies to EBV encoded dUTPase and the EBV DNA encoded polymerase antigens and then assayed by both Luminex and ELISA.

### Analytical Specificity

Analytical specificity will be studied by spiking pooled negative patient specimens with antibodies to EBV encoded dUTPase and the EBV DNA encoded polymerase gene product.

### Precision

For precision six specimens will be tested. On each day of testing, each sample will be diluted twice and then loaded for four replicates resulting in a total of eight wells of each of the six samples. This protocol will be followed for three days. Selection of specimens will be done such that some of them will be clearly negative, some will be clearly positive and some will be weakly positive or just near the cutoff of the assay. These results will then be used to calculate mean U/mL values, standard deviations, and percent CV.

### Summary of EBV Assay Development

A multiplex assay will be developed using specific antigens as listed above. Microsphere Luminex technology will be used for this assay. Microsphere or beads will be coated with specific antigens as listed above and then optimized and validated in singlet and multiplexes. Optimization and validation will be performed using banked human subject specimens that will be diluted to note detection to establish cutoff and reference ranges.

### Method of Treatment

In another aspect, the invention provides methods of treating the EBV subset of ME/CFS patients. In some embodiments, the methods of treating a patient with CFS involves evaluating the patient for serologic evidence of the presence of nucleic acid molecules that indicate primary infection by one or more CFS-causing agents, thereby detecting the presence of each CFS-causing agent present in the patient; evaluating the patient for serologic evidence of one or more co-infection; determining whether one or more co-infections are present in the patient; administering, or causing to be administered, to a patient a therapeutically effective amount of at least one pharmaceutical composition; further comprising at least one antiviral agent such that each CFS-causing agent found in the patient is effectively treated by at least one antiviral agent administered to the patient; and, if one or more co-infections are present, also administering, or causing to be administered, to the patient therapeutically effective amount of at least one pharmaceutical composition such that each co-infection found in the patient is effectively treated by at least one pharmaceutical composition administered to the patient, thereby treating the CFS. Each pharmaceutical composition can comprise one active agent, or it can comprise a cocktail of more than one active agent. The co-infection can be with, for example, Borrelia burgdorferi, Streptococcus pyogenes, Ehrlichia chaffeensis, Babesia microti and Mycoplasma pneumonia. The antiviral agent can be, for example, valacyclovir, valganciclovir, maribavir, famciclovir and foscarnet. However, any antiviral agent that is effective against a CFS-inducing infection can be used according to the methods disclosed herein.

Group A comprises CFS patients with EBV, HCMV, and/or HHV6 persistent infection in single virus or combination, but without additional co-infections. The following are the criteria for selecting these patients;

1) Patients meet international and CDC criteria for CFS and have abnormal 24 hour ECG monitors (as determined by the presence of tachycardia and/or abnormal T waves).

2) Patients are positive for HCMV, EBV and/or HHV6, as determined by detection of serum antibodies to each of these virus's or to gene products of these virus's, weather detected using enzyme-linked immunosorbent assay (ELISAN) or other methods, including those of the invention. 3) Patients are negative for CFS co-infections, such as for example Borrelia burgdorferi, Streptococcus pyogenes, Ehrlichia chaffeensis, Babesia microti and Mycoplasma pneumonia. These patient respond favorably, there are validated energy index point scores increase from less than to greater than 6, usually reaching an energy point score (EI) of 7-9 within six to twelve months antiviral therapy with appropriate antiviral agents, including, for example valacyclovir, valganciclovir, maribavir, cidofovir, famciclovir and foscarnet.

Group B comprises CFS patients with EBV, HCMV and/or HHV6 persistent infection, either alone or in combination, but also having one or more co-infections. The following are the criteria for group B patients;

1) Patients meet international and CDC criteria for CFS and have abnormal 24 hour ECG monitors (as determined by the presence of tachycardia and/or abnormal T waves).

2) Patients are positive for HCMV, EBV and/or HHV6 infection. 3) Patients are positive for one or more of the following: Borrelia burgdorferi, Streptococcus pyogenes, Ehrlichia chaffeensis, Babesia microti and Mycoplasma pneumonia.

CFS patients in group B can be treated with for example, valacyclovir, valganciclovir, maribavir, or other derivatives or benzimidazole, as appropriate to the particular infections present in a particular patient. famciclovir and foscarnet. CFS patients in group B can also be treated with appropriate therapy for co-infection, which may include, for example infection with Borrelia burgdorferi, Streptococcus pyogenes, Ehrlichia chaffeensis, Babesia microti and Mycoplasma pneumonia. Generally, unless group B patients are treated for the co-infection they will not improve.

Infection with Borrelia burgdorferi can be diagnosed by, for example, detecting the presence of IgM or IgG to Borrelia burgdorferi using Western blot or ELISA. It may be possible that IgG is not detected in a patient with CFS, in which case a positive IgM result would be diagnostic of infection with Borrelia burgdorferi. Antigens used for this assay are exact prototypes used by US Centers for Disease Control.

Infection with Borrelia burgdorferi can be treated with, for example, intravenous (IV) ceftriaxone 0.1-5 gm, for example 1.0-1.5 gm, intravenous piggy-back (IVPB) every 12 hours for 30 days, followed by oral amoxicillin 0.01-4.0 gm, for example 0.5-0.75 gm, 4 times/day until above serum tests negative. IV penicillin G or its equivalent every 6-8 hours can substitute for ceftriaxone. For treatment of Borrelia burgdorferi, as well as of any other secondary infection described herein, qualified health care personnel can prescribe appropriate dosages for effective treatment of CFS. Any dosage that falls within the scope of sound medical judgment is contemplated as part of this invention.

Adult rheumatic fever is caused by a hyperimmune response to, for example, Streptococcus pyogenes infection. It can be diagnosed by, for example, finding an elevated antistreptolysin O (ASO) titer (LabCorp, Dublin, Ohio); for example, an ASO titer over 400 units would be diagnostic of adult rheumatic fever or co-infection by Streptcoccus pyogenes. Adult rheumatic fever may also be accompanied by, for example, thickening of the aortic and/or mitral valve, which can be viewed on an echocardiogram.

Adult rheumatic fever can be treated with, for example, ceftriaxone or penicillin G as above followed by bicillin 0.1-5 Mu, for example 1.2 Mu, every 2-4 weeks until ASO titer is less than 200. A CatScan of sinuses/mastoids may be indicated to exclude obstructive sinusitis. Bicillin may be necessary for 2-4 years.

Babesiosis can be diagnosed by, for example, finding an elevated serum titer for IgG to Babesia microti. Chronic Babesiosis can be treated with, for example, Ataquavone 100-1500, for example 750 mg orally, plus azithromycin 0.01-4 gm, for example 0.5 gm, twice daily for 6 weeks. Detection of IgM to Babesia microti can also be done.

Ehrlichia chaffeensis co-infection can be determined by, for example, detecting a positive serum titer for IgG or IgM to Ehrlichia chaffeensis. Chronic Ehrlichiosis can be treated with, for example, IV doxycycline 10-1000 mg, for example 100 mg, every 12 hours for 4, 6, or 12 weeks.

Infection by Mycoplasma pneumoniae can be diagnosed by, for example, finding a markedly positive serum titer for IgG or IgM to Mycoplasma pneumoniae. Infection by Mycoplasma pneumoniae may also be accompanied by, for example, an abnormal standard 12-lead electrocardiogram.

Mycoplasma pneumoniae myocarditis can be treated with, for example, IV doxycycline plus/minus IV azithromycin for 6 weeks.

Effective sublassifications of CFS patients have not been previously recognized. It is further believed that chronic fatigue syndrome may manifest in conjunction with other diseases and as such, with the identification of a biomarker to confirm the presence of Group A EBV ME/CFS, patients that exhibit fatigue syndromes in conjunction with other illnesses can now be tested and treated for the accompanying ME/CFS manifestations.

### Pharmaceutical Compositions and Administration

In general, to provide a therapeutically effective amount of the antiviral agent, a suitable effective dose will be in the range of 0.1 to 20 grams a day and preferably in the range between 0.3 to 15 grams per day, more preferably about 0.5 to 10 grams per day. The dosage of course, varies with the body weight of the patient up to a 70 kg individual, a dose of 4 grams per day may be appropriate (e.g., 10 mg per KG valacyclovir every six hours). The desired dose can be presented as two-four or more smaller doses administered at appropriate intervals throughout the day. These smaller doses may be administered in unit's dosage forms. For example, for valacyclovir and famciclovir, the dosage can be, for example 14 mg/kg every 6 hours (1.0 g every 6 hours for a 70 kg person). The dosage of valacyclovir and famciclovir can be, for example, up to or at least about 0.5-8 grams every 6 hours. The dosage of valgancyclovir can be, for example, from about 450-900 mg every 12 hours, or up to or at least about 100-2000 mg or more every 12 hours, depending on for example patient weight and tolerance. For maribavir the dosage can be, for example, for about 400-500 mg every 8 hours or for example up to or at least about 100-1000 or more every 8 hours. As cidofovir is administered intravenously, it is two to three time more effective than other antiviral agents. Accordingly, the dosage is adjusted to 3-5 mg/ml every two weeks. In addition to the antiviral agent, two other medications are preferably administered to prevent side effects. In one preferred embodiment, Probenecid is administered along with the antiviral agent to inhibit tubular secretions. In another preferred embodiment, Zofran is administered along with the antiviral agent to prevent nausea. In yet another preferred embodiment, both Probenecid and Zofran are administered with the antiviral agent. Qualified health care personnel can prescribe appropriate dosages for effective treatment of CFS. Any dosage that falls within the scope of sound medical judgment is contemplated as part of this invention.

For those patients that are over 50 and/or have had CFS symptoms or complications for over 10 years, cidofovir is a preferred antiviral agent. For such patients, cidofovir reduces the treatment time from over one year to potentially in the range of 6-9 months. For those patients that experience gastrointestinal problems with valacyclovir, famciclovir can serve as a suitable option.

In particular for valacyclovir, or a derivative such valacyclovir hydrochloride, a patient can be administered a dosage in the range of 0.1 to 50 mg/kg of body weight of the patient per dosing interval, generally every 6 hours. The dosing interval is determined by the bioavailability of the antiviral agent and its excretion from the body. For example, the patient can be administered a dosage in the range of 0.3 to 40 mg/kg of body weight of valacyclovir hydrochloride orally every 6 hour. For example, a patient can be administered 10 mg/kg of body weight valacyclovir hydrochloride every 6 hours.

The treatment period for a CFS patient varies on a case-by-case basis. It is believed that for some, CFS is an ongoing and persistent problem requiring continued treatment. The duration of the therapy depends on the intensity of the CFS as affected by the therapy. One indicator of an improvement in EBV-isolated CFS patients is a decrease of a level of IgM antibodies to viral capsid antibodies (VCA) for EBV. Generally, the therapy duration is proportional to the intensity and duration of the CFS manifestation. Accordingly, following administration of an antiviral agent, supplemental tests are helpful to check for recurrent CFS and to determine the treatment duration. The duration of treatment may be 6-18 months or longer or shorter as determined by the attending physician using the methods described herein. Notably, the shorter the duration of CFS, the shorter the treatment period.

Antiviral agents which demonstrate anti-herpetic action, such as those specific to, for example, EBV, HCMV, or HHV6, can be used for the treatment of chronic fatigue syndrome. Such antiviral agents may be effectively administered, for example, by oral methods, or as larger doses in time delay formulations. Included among this group of antiviral agents are valacyclovir, valganciclovir, maribavir, famciclovir and foscarnet and other herpetic antiviral agents and pharmaceutically acceptable derivatives of these antiviral agents. Such pharmaceutically acceptable derivatives include salts, hydrolysable esters and chelates of the antiviral agents and such similar derivatives which have no negative pharmaceutical effect on the patient upon administration and are thus “pharmaceutically acceptable”. A pharmaceutically acceptable salt can become a for example, an acidic salt derived from an appropriate acid, for example hydrochloric, sulfuric, phosphoric, maleic, fumaric, citric, lactic, tartaric, acetic or p-toluenesulphonic acid. For Group A patients, acylocivir is typically not absorbed.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A method of detecting Abortive lytic replication-based Epstein-Barr Virus in a biological sample of a patient, comprising:
detecting the presence of IgG serum antibodies to the EBV Early encoded protein deoxyribonucleotide polymerase accessory processivity factor, BMRF1; and
determining EBV-based SEID in a patient with the presence of antibody titers over 10.0.

2. The method of claim 1, wherein the step of detection is conducted using a fluorescence assay.

3. The method of claim 2, wherein the presence of BMRF1 is detected in the assay with the use of fluorescent signals to monitor the spectral properties of the biological sample to determine the presence of BMRF1.

4. The method of claim 3, further comprising the steps of:
coupling BMRF1 to a bead; diluting the biological sample and testing the biological sample for a fluorescence signal that is positive for BMRF1 to detect the presence of Abortive lytic replication-based Epstein-Barr virus.

5. The method of claim 4, further comprising the step of coupling BMRF1 to a bead in region 13.

6. A method of treating Epstein-Barr Virus abortive lytic replication in a patient, comprising the steps of;
identifying that the patient falls within Group A EBV patients;
obtaining a biological sample from the patient and confirming the presence of BMRF1 titers; and
treating the patient with an effective amount of at least one antiviral agent if the presence of BMRF1 titers above 10.0 are confirmed in the patient's biological sample.

7. The method of claim 6, further comprising the step of treating the patient with a dosage of 0.3 to 15 grams of antiviral agent per day.

8. The method of claim 6, further comprising the step of treating the patient with a dosage of 0.1 to 20 grams of antiviral agent per day.

9. The method of claim 6, further comprising the step of treating the patient with a dosage of 0.5 to 10 grams of antiviral agent per day.

10. The method of claim 6, further comprising the step of treating the patient with an antiviral agent selected from the group consisting of valacyclovir, valganciclovir, maribavir, cidafovir, famciclovir and foscarnet.

11. The method of claim 6, further comprising the step of treating the patient with Probenecid.

12. The method of claim 6, further comprising the step of treating the patient with Zofran.

13. The method of claim 6, wherein the antiviral agent is administered for a period of over 10 months.

14. The method of claim 6, wherein the antiviral agent is administered for a period of over 12 months.

15. The method of claim 6, where the antiviral agent is administered until testing of the patient reveals low-titers of EBV EA(D), in the range of 40 units or less.

16. The method of claim 6, wherein the patient is tested monthly for the treatment period to test the EBV EA(D) titer levels.

17. The method of claim 6, further comprising testing the patient to confirm the absence of co-infections.

18. The method of claim 6, further comprising screening the patient for CFS criteria including the use of ECG monitors.

19. The method of claim 6, further comprising the step of treating a patient above the age of 60 with cidofovir.

20. A kit for detecting abortive lytic replication-based EBV in a biological sample of an SEID patient which comprises an antibody that binds specifically to the BMRF1 protein, wherein said binding characteristics comprise streptavidin-phycoerythin binding to an IgG antibody that is bound to the BMRF1 protein together with reagents for the detection of binding of said antibody to the BMRF1 protein.
